(19) United States
(12) Patent Application Publication
(10) Pub. No.: US 2024/0245624 A1
(43) Pub. Date: Jul. 25, 2024

(54) COMPOSITIONS AND METHODS FOR IMPROVING SKIN BARRIER

(71) Applicant: LYCORED LTD., Be’er Sheva (IL)

(72) Inventor: Nava DAYAN, Fair Lawn, NJ (US)

(21) Appl. No.: 18/564,091

(22) PCT Filed: Jun. 16, 2022
(86) PCT No.: PCT/IL2022/050650

§ 371 Date: Nov. 26, 2023

Related U.S. Application Data
(60) Provisional application No. 63/211,072, filed on Jun. 16, 2021.

(51) Int. Cl. A61K 31/01; A61K 31/355; A61K 31/575; A61P 17/00

(52) U.S. Cl. CPC A61K 31/01; A61K 31/355; A61K 31/575; A61P 17/00

(57) ABSTRACT

The present invention is directed to a composition comprising phytoene, phytofluene, and zeta carotene, and methods of using the same, such as for improving skin barrier functionality.

### CROSS-REFERENCE TO RELATED APPLICATIONS

This application claims the benefit of priority of U.S. Provisional Patent Application No. 63/211,072, titled “COMPOSITIONS AND METHODS FOR IMPROVING SKIN BARRIER”, filed 16 Jun. 2021, the contents of which are incorporated herein by reference in their entirety.

### FIELD OF THE INVENTION

The invention relates generally to the field of carotenoids, and methods of using same, such as for improving skin barrier.

### BACKGROUND

The tomato (Solanum lycopersicum) is a fruit that is eaten throughout the world. Tomatoes are rich in nutrients including vitamin C, potassium, essential amino acids and various antioxidants. Tomatoes are the major dietary source of the potent antioxidant lycopene. Tomato consumption was demonstrated to provide protection against oxidative stress-related diseases including cancer and cardiovascular disease.

The use of dietary supplements has been increasing worldwide. A survey of consumers in the United States covering 2015-2019 revealed upward trends in the use of various supplements. Among the most used supplements are, in order of decreasing use: fish oil, glucosamine/chondroitin, probiotics/prebiotics, melatonin, coenzyme Q10, echinacea, cranberry pills, garlic extract, ginseng and gingko biloba. Some of these supplements have demonstrated health benefits, while others are controversial.

Lumenato™ is a dietary supplement prepared from dried pulp of yellow tomatoes. It is rich in carotenoids including lycopene (0.5 mg/capsule), 15-cis-phytoene (7.33 mg/capsule), phytofluene (2.06 mg/capsule), β-carotene (0.18 mg/capsule), γ-carotene (0.03 mg/capsule), and ζ-carotene (2.27 mg/capsule), and tocopherols (1.14 mg/capsule). In general, the carotenoids and tocopherols are antioxidants that provide numerous health benefits.

Experiments conducted in the past two decades to study potential effects of oral intake of carotenoids on skin focused mainly on the effect of β-carotene and lycopene with scarce information on phytoene and phytofluene. Specific benefits of lycopene and/or β-carotene include protection against UV-induced erythema and sun damage, reduced oxidative stress, and antioxidant/anti-inflammatory activities. β-Carotene is, of course, the precursor of vitamin A, and so supports collagen synthesis. Lycopene has been shown to protect against UV damage to cultured human dermal fibroblasts. In addition, it has been shown to suppress chemically induced skin cancer in an animal model. This as well as its ability to ameliorate DNA damage induced by 4-hydroxyestradiol are thought to be attributable to its antioxidant properties. In addition to antioxidant activity, phytoene and phytofluene are colorless carotenoids that absorb ultraviolet light. This property could provide an additional level of protection to the skin.

The human skin is exposed to various sources of oxidative stress including ultraviolet light, ozone and other air pollutants and metabolism of resident microorganisms. These stressors accelerate skin aging resulting in wrinkles, reduced elasticity and a rough texture.

Fibroblasts are the most abundant cells in the dermis. They synthesize collagen and maintain the extracellular matrix. During aging, collagen fibrils are progressively lost leading to wrinkling. Fibroblasts play significant roles in innate immunity of the skin by producing cytokines to attract leukocytes to the dermis. Fibroblasts also communicate with melanocytes in the basal layer of the epidermis to influence melanin synthesis. Keratinocytes are the main cell type in the epidermis. They differentiate to produce a stratum corneum consisting of flattened, keratin-filled cells surrounded by a lipid matrix. The stratum corneum provides the permeability barrier of the skin. It serves to limit loss of water and electrolytes as well as limiting the penetration of pollutants and harmful chemicals, bacteria, and viruses into and through the skin. The lipids of the stratum corneum consist mainly of ceramides, cholesterol and long chain fatty acids. One of the ceramides, the acylceramide, is quite unusual in that it contains 30- through 34-carbon long ω-hydroxyacids amide-linked to a long-chain base with linoleic acid ester-linked to the ω-hydroxyl group. This linoleate-containing lipid is essential for the organization of the lipids and is essential for barrier function.

There is still a great need for compositions and methods for reducing transepidermal water loss (TEWL), and therefore increase and/or improve skin barrier.

### SUMMARY

The following embodiments and aspects thereof are described and illustrated in conjunction with systems, tools and methods which are meant to be exemplary and illustrative, not limiting in scope.

The present invention, in some embodiments, is based, in part, on the surprising findings that consumption of a particular carotenoids composition improved skin barrier, as demonstrated by TEWL reduction.

According to a first aspect, there is provided a method for treating a skin barrier related disease or disorder in a subject in need thereof, comprising the step of administering to the subject a therapeutically effective amount of a composition comprising: phytoene in the amount of 55-65% (w/w) of the total carotenoids in the composition, phytofluene in the amount of 10-20% (w/w) of the total carotenoids in the composition, zeta carotene in the amount of 15-25% (w/w) of the total carotenoids in the composition, thereby treating a skin barrier related disease or disorder in the subject.

According to another aspect, there is provided a composition comprising: phytoene in the amount of 55-65% (w/w) of the total carotenoids in the composition, phytofluene in the amount of 10-20% (w/w) of the total carotenoids in the composition, zeta carotene in the amount of 15-25% (w/w) of the total carotenoids in the composition, for use in the treatment of a skin barrier related disease or disorder in a subject in need thereof.

In some embodiments, the method further comprises a selecting step preceding the administering step, comprising determining a transepidermal water loss (TEWL) value of a skin of the subject, wherein a TEWL value of at least 12 (g·h/m²) is indicative of the subject being suitable for the treating.

In some embodiments, the TEWL value is determined for a facial skin, forehead skin, a forearm skin, hand skin, palm skin, leg skin, elbow skin, or any combination thereof, of the subject.

In some embodiments, the skin barrier related disease is an epidermal skin disease or disorder.

In some embodiments, the skin barrier related disease is characterized by alteration of the stratum corneum: lipid content, oxidative state, arrangement, or any combination thereof.

In some embodiments, the skin barrier related disease is characterized by a reduced amount of linoleate-containing acylceramide.

In some embodiments, the skin barrier related disease is selected from the group consisting of: atopic dermatitis, psoriasis, type 2 Gaucher syndrome, Sjogren-Larsson syndrome, lamellar ichthyosis, X-linked ichthyosis, bullous ichthyosiform erythroderma, essential free fatty acid deficiency, acne vulgaris, aged dry skin, hypohidrotic ectodermal dysplasia, and atopic eczema.

In some embodiments, treating comprises reducing: TEWL, number of pores, area covered by pores, level of redness, area covered by redness, roughness, or any combination thereof, of a skin of the subject.

In some embodiments, reducing is by at least 5% compared to a control.

In some embodiments, the weight ratio of the phytoene and the phytofluene combined to the zeta carotene ranges from 15:1 (w/w) to 2:1 (w/w).

In some embodiments, the composition further comprises an additional carotenoid selected from the group consisting of: lycopene, beta carotene, gamma carotene, and any combination thereof.

In some embodiments, the composition comprises the lycopene in the amount of less than 5% (w/w) of the total carotenoids in the composition, the beta carotene in the amount of less than 5% (w/w) of the total carotenoids in the composition, the gamma carotene in the amount of 0.2-1.5% (w/w) of the total carotenoids in the composition, or a combination thereof.

In some embodiments, the composition comprises a total carotenoid amount of 10-15% (w/w) of the composition.

In some embodiments, the composition further comprises a tocopherol.

In some embodiments, the composition comprises the tocopherol in the amount of 10-30% (w/w) total carotenoids in of the composition.

In some embodiments, the composition further comprises a phytosterol.

In some embodiments, the composition comprises the phytosterol in the amount of 5-15% (w/w) total carotenoids in of the composition.

In some embodiments, administering comprises orally administering, topically administering, or both.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

In addition to the exemplary aspects and embodiments described above, further aspects and embodiments will become apparent by reference to the figures and by study of the following detailed description.

### BRIEF DESCRIPTION OF THE FIGURES

FIG. 1 includes a graph showing the effect of a golden tomato extract (GTE, also referred to herein as “Lumenato™”) on the transepidermal water loss (TEWL). A, High TEWL-GTE (N=18, one subject missed the 8-week clinic visit, 4 weeks p=0.428, 8 weeks p=0.11, 16 weeks p<0.001); B, High TEWL-Placebo (N=14, one subject missed the 8-week clinic visit, 4 weeks p=0.382, 8 weeks p=0.506, 16-week p=0.293); C, Low TEWL-GTE (N=14, 4 weeks p=0.129, 8 weeks p=0.49, 16 weeks p=0.421); and D, Low TEWL-Placebo (N=15, one subject missed the 8- and 16-week clinic visits, 4 weeks p=0.099, 8 weeks p=0.838, 16 weeks p=0.231).

FIG. 2 includes images of a front standard view “before” and “after” treatment of a panelist with 53% improvement in TEWL.

FIGS. 3A-3C include images of a panelist “before” and “after” treatment showing 26% reduction in TEWL. (3A) front; (3B) left; and (3C) right. Red areas are highlighted.

FIG. 4 include images of a left view “before” and “after” treatment of a panelist with 33% improvement in TEWL analysis of pores.

FIG. 5 include images of a right view “before” and “after” treatment of a panelist with 48% improvement in TEWL analysis of pores.

FIGS. 6A-6B include images of left (6A) and right (6B) views “before” and “after” treatment of panelists with 21 and 33% improvement in TEWL analysis of texture, respectively.

FIG. 7 includes a scheme of a non-limiting theory of the two mechanisms by which the golden tomato extract disclosed herein strengthens the physical and the biological barrier of the skin.

FIGS. 8A-8F include vertical bar graphs showing the average scores of different parameters before and after 12-week of oral administrations of Lumeneto capsules. As observed in (8B) there was a highly significant (p<0.001) improvement in skin elasticity (21.54%) and firmness (27.18%) after using the supplement for 12 weeks. (8A) Shows various measurements of skin tonality. As observed in the graph, although there was no improvement in pigmented spots, there was still a 21% improvement in evenness of skin (p<0.001)=. The skin appeared 29.75% brighter (p<0.001), and dark spots of the face reduced by 10.96% (p<0.001). Periorbital dark circles also appeared to diminish by 17.9% (p<0.001). (8C) Displays that there was 21.6% (p<0.001) perceived improvement in skin hydration and 18.18% improvement in skin texture (p<0.001) after 12 weeks of using the supplement. (8D) Shows that although there was no improvement in deep wrinkles, there was a significant improvement of 12.97% (p<0.001) in fine lines and wrinkles of the face after 12 weeks of supplement use. As observed in (8E) there was a slight improvement in the tendency of the skin to develop redness. The general look of aged/unhealthy skin reduced significantly (p<0.001) after using the supplement with a 23.53% reduction. (8F) Shows the average score of overall skin condition before and after 12-week treatment of the supplement. As observed in the graph, there was a highly significant (p<0.001) improvement of 28.22% in the overall skin condition after using the supplement.

FIGS. 9A-9E include graphs showing results from FACE-QTM Satisfaction with Skin Scale before treatment and after 4, 8, and 12 weeks of oral administrations of Lumeneto capsules and again after treatment was stopped for 2 weeks. The data presented are from satisfaction score where score 1 was very dissatisfied, 2 was somewhat dissatisfied, 3 was somewhat satisfied, and 4 was very satisfied. The number of subjects reporting a score of 3 and 4 was counted and expressed in the graph as percent of population showing approval. (9A) Shows “how does your facial skin look” first thing in the morning and at the end of the day. Initially, the score was at the edge of satisfied and dissatisfied; however, after treatment, there was a steady improvement up to eight weeks of 84%-75% (p<0.001), after which there was a levelling off. The effect retained after the treatment was discontinued for two weeks. (9B) Shows a similar trend for perception of healthy and attractive look of the face. For both these parameters, there was steady improvement over the course of 12-week treatment at which point there was a statistically significant (p<0.001) improvement of 74.6% and 65.08% (p<0.001) for healthy and attractive skin, respectively. These perceptions continued to improve despite discontinued treatment for 2 weeks. (9C) Perception of refreshed and radiant face. For these two parameters, there was steady improvement over the course of 12 weeks of treatment at which point there was a statistically significant (p<0.001) change as compared to baseline. These perceptions continued to improve despite discontinued treatment for 2 weeks. (9D) Facial skin tonality. After treatment there was a steady improvement up to eight weeks of 61.9%-74.6% (p<0.001) in evenness of color and tonality. By the 12th week of treatment over 71% (p<0.001) of the population professed that the treatment was effective for skin tonality and evenness of tone. After treatment was discontinued for two weeks, there was a further improvement of 81% and 72.41% in evenness of skin tone and tonality, respectively. (9E) Shows that 38.71% of the population noticed a reduction in skin redness after using the supplement for 4 and 8 weeks, increasing to 41.94% after 12 weeks of supplement use. Improvement in the look and feel of healthy skin was observed by 35% of the population after 4 weeks of use increasing to 56.45% and 58.06% after 8- and 12-week use, respectively. The overall appearance of skin improved in 35.48% of the subjects after 4 weeks of use increasing to 66.13% and 62.9% after 8 and 12 weeks, respectively.

### DETAILED DESCRIPTION

In some embodiments, the present invention is directed to a composition comprising a plurality of carotenoids, and methods of using same. The present invention is based, in part, on the surprising findings that a tomato extract comprising high amounts of phytoene, phytofluene, and zeta carotene, improves skin barrier and integrity in two complementary modalities: biological (innate immunity) and physical (the stratum corneum barrier).

### Method of Treatment

According to some embodiments, there is provided a method for treating impaired skin barrier function in a subject in need thereof, comprising the step of administering to the subject a therapeutically effective amount of the herein disclosed composition.

In some embodiments, the subject in need of treatment is a healthy subject being afflicted with impaired barrier function. In some embodiments, the subject in need of treatment is a healthy subject being afflicted with impaired skin barrier.

In some embodiments, treating comprises improving skin barrier or barrier function, in the subject.

In some embodiments, treating comprises reducing TEWL in the subject.

According to some embodiments, there is provided a method for treating a skin barrier related disease or disorder in a subject in need thereof, comprising the step of administering to the subject a therapeutically effective amount of the herein disclosed composition, thereby treating a skin barrier related disease or disorder in the subject.

In some embodiments, a skin barrier disease or disorder comprises impaired barrier function or impaired skin barrier.

In some embodiments, the composition comprises: phytoene in the amount of 55-65% (w/w) of the total carotenoids in the composition, phytofluene in the amount of 10-20% (w/w) of the total carotenoids in the composition, zeta carotene in the amount of 15-25% (w/w) of the total carotenoids in the composition.

In some embodiments, the method further comprises a selecting step comprising determining a transepidermal water loss (TEWL) value of a skin of the subject.

In some embodiments, the selecting step precedes the administering step.

In some embodiments, a TEWL value of at least 1 (g·h/m²), at least 2 (g·h/m²), at least 4 (g·h/m²), at least 5 (g·h/m²), at least 7 (g·h/m²), at least 10 (g·h/m²), at least 11 (g·h/m²), at least 12 (g·h/m²), at least 13 (g·h/m²), at least 14 (g·h/m²), at least 15 (g·h/m²), at least 16 (g·h/m²), or at least 20 (g·h/m²), is indicative of the subject being suitable for treating according to the herein disclosed method, or any value and range therebetween. Each possibility represents a separate embodiment of the invention. In some embodiments, a TEWL value of 10-14 (g·h/m²), 1-15 (g·h/m²), 2-8 (g·h/m²), 3-12 (g·h/m²), 5-13 (g·h/m²), 4-10 (g·h/m²), 10-18 (g·h/m²), 11-20 (g·h/m²), 13-17 (g·h/m²), or 12-20 (g·h/m²), is indicative of the subject being suitable for treating according to the herein disclosed method. Each possibility represents a separate embodiment of the invention.

In some embodiments, a subject suitable for treatment according to the method of the invention is characterized by having compromised and/or impaired barrier function and/or impaired skin barrier.

In some embodiments, a TEWL value of at least 10 (g·h/m²), 12 (g·h/m²), 14 (g·h/m²), 17 (g·h/m²), or 20 (g·h/m²), is indicative of compromised and/or impaired barrier function and/or impaired skin barrier, or any value and range therebetween. Each possibility represents a separate embodiment of the invention. In some embodiments, a TEWL value of 12-20 (g·h/m²), 11-18 (g·h/m²), 12-19 (g·h/m²), or 10-20 (g·h/m²), is indicative of compromised and/or impaired barrier function and/or impaired skin barrier. Each possibility represents a separate embodiment of the invention.

In some embodiments, the TEWL value is determined for a facial skin, forehead skin, a forearm skin, a volar forearm skin, hand skin, palm skin, leg skin, elbow skin, or any combination thereof, of the subject. In some embodiments, the TEWL value is determined for a facial skin of the subject. In some embodiments, a facial skin comprises a cheek of the subject.

As used herein, the terms “transepidermal water loss” or “TEWL” refer to the loss of water from inside the body through the epidermis to the surrounding environment via diffusion and evaporation. The ability to measure the TEWL is useful in detecting damage to the skin, e.g., indicative of a barrier disease or disorder.

Methods and device suitable for determining a skin TEWL are common and would be apparent to one of ordinary skill in the art, and review, for example by Sotoodian and Maibach, 2012 (Clinics in Dermatology).

In some embodiments, the subject is characterized or afflicted by at least one of: poor nutrition, reduced blood supply, exposure to radiation, oxidative stress, immunocompromising, emotional stress, skin dryness, affliction of a chronic inflammatory disease, genetic disposition, affliction of skin dysbiosis, reduced hygiene, excessive hygiene, reduced skin lipid content, sebum content, or both, and any combination thereof.

In some embodiments, radiation comprises any radiation wavelength within the light spectrum. As used herein, the term “light spectrum” encompasses wave lengths ranging from 10-9 m to 10-3 m. In some embodiments, radiation wavelength within the light spectrum comprises UV radiation, visible light radiation, infrared radiation, or a combination thereof. In some embodiments, an exposure to radiation comprises an exposure to sunlight.

As used herein, the term “ultraviolet (UV)” encompasses any wavelength of the UV range. In some embodiments, UV is UV radiation. In some embodiments, UV radiation is UVA radiation, UVB radiation, UVC, or any combination thereof.

In some embodiments, chronic inflammatory disease comprises a skin disorder selected from: acne, psoriasis, atopic dermatitis, contact dermatitis, and rosacea.

In some embodiments, chronic inflammatory disease comprises a systemic inflammatory disease.

In some embodiments, the skin barrier related disease is an epidermal skin disease or disorder. In some embodiments, the skin barrier related disease is characterized by or comprises aberrations, deformations, pathologies, or any combination thereof, of the epidermal layer of the skin. In some embodiments, the skin barrier related disease comprises or is characterized by alteration of the stratum corneum layer of the epidermis. In some embodiments, the skin barrier related disease comprises or is characterized by alterations or modifications of the stratum corneum layer: lipid content, oxidative state, arrangement, or any combination thereof.

In some embodiments, alterations or modifications of the lipid content of the stratum corneum layer are such that reduce the functionality of the stratum corneum layer, e.g., impair or reduce skin barrier activity.

In some embodiments, the skin barrier related disease is characterized by a reduced amount of linoleate-containing acylceramide.

In some embodiments, a skin barrier related disease is selected from: xerosis, atopic dermatitis, contact dermatitis, psoriasis, type 2 Gaucher syndrome, Sjogren-Larsson syndrome, lamellar ichthyosis, X-linked ichthyosis, bullous ichthyosiform erythroderma, essential free fatty acid deficiency, acne vulgaris, aged dry skin, hypohidrotic ectodermal dysplasia, atopic eczema, or any combination thereof.

As used herein, the term “skin barrier related disease or condition” encompasses any disease or condition wherein impaired skin barrier, disrupted skin barrier, non-functional skin barrier, dysfunctional skin barrier, or any combination thereof, initiates, stimulates, propagates, increases, enhances, any equivalent thereof, or any combination thereof, a pathological state or divergence from homeostasis in the subject. In some embodiments, skin barrier related disease comprises any disease or condition involving skin barrier impairment, disruption, non-functionality, dysfunctionality, reduced or inhibited functionality, as part of the disease or condition pathogenesis and/or pathophysiology.

In some embodiments, treating comprises reducing: TEWL, number of pores, area covered by pores, level of redness, area covered by redness, roughness, or any combination thereof, of a skin of a subject.

In some embodiments, reducing is by at least 5%, at least 15%, at least 25%, at least 35%, at least 50%, at least 75%, at least 85%, at least 95%, at least 97%, at least 99%, or 100% reduction, or any value and range therebetween. Each possibility represents a separate embodiment of the invention. In some embodiments, reducing is 5-50%, at least 1-25%, 10-55%, 20-95%, 50-99%, 40-75%, 5-85%, 30-95%, or 25-100% reduction. Each possibility represents a separate embodiment of the invention.

As used herein, the terms “subject” or “individual” or “animal” or “patient” or “mammal,” refers to any subject, particularly a mammalian subject, for whom therapy is desired, for example, a human.

In some embodiments, the subject is at increased risk of developing acne. In some embodiments, the subject has increased predisposition to developing acne. In some embodiments, the subject is characterized by having skin being prone to developing acne.

In some embodiments, reducing is compared to a control.

The term “control” as used herein is interchangeable with “benchmark” or “baseline”.

As used herein, a control comprises the skin of a healthy subject. In some embodiments, the control is a healthy skin sample derived, isolated, or obtained from the same subject (e.g., the subject afflicted with the skin barrier related disease, such that may include, but not limited to a skin comprising patches with confined areas of compromised barrier). In some embodiments, a control comprises a healthy skin. In some embodiments, a healthy skin comprises a skin of a healthy subject. In some embodiments, a healthy skin comprises a skin of the same subject prior to the subject being afflicted with a skin barrier related disease, as disclosed herein.

In some embodiments, administering comprises orally administering. In some embodiments, administering comprises topically administering. In some embodiments, administering comprises combined orally administering and topically administering.

As used herein, the phrase “inflammatory disease” refers to any disease involving a multicomponent response of an organism (e.g., cells of the immune system, molecular signal mediators such as cytokines, etc.) to a harmful exogenous entity, for example, bacteria, fungi, viruses, protozoa, allergens, pollutants, etc.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described compositions or formulations prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

In some embodiments, preventing comprises reducing the disease severity, delaying the disease onset, reducing the disease cumulative incidence, or any combination thereof.

According to some embodiments, there is provided a method for modifying the expression level of a gene in a cell, comprising contacting the cell with composition of the invention, thereby modifying the expression of the gene.

In some embodiments, the gene is selected from: IL6, CCL2, CXCL1, TNFPI3, DGAT2L6, ALOX5, BACH2, CYP4F11, CYP4A22, B3GALT1, B3GALT2, OR52N, OR5D15P, OR52B6, OR1F1, OR2L3, OR2V2, OR51AB1P, OR1B1, OR4C12, OR6C76, OR2AH1P, OR7A18P, OR1N2, OR10B1P, OR11L1, OR5BA1P, OR5M11, OR9A4, OR4U1P, or any combination thereof.

In some embodiments, the gene is selected from: IL6, CCL2, CXCL1, TNFPI3, DGAT2L6, ALOX5, BACH2, CYP4F11, CYP4A22, B3GALT1, B3GALT2, or any combination thereof.

In some embodiments, the gene is selected from: OR52N, OR5D15P, OR52B6, OR1F1, OR2L3, OR2V2, OR51AB1P, OR1B1, OR4C12, OR6C76, OR2AH1P, OR7A18P, OR1N2, OR10B1P, OR11L1, OR5BA1P, OR5M11, OR9A4, OR4U1P, or any combination thereof.

In some embodiments, modifying comprises increasing or decreasing.

In some embodiments, modifying comprises enhancing or reducing.

In some embodiments, the method comprises increasing the expression level of: IL6, CCL2, CXCL1, TNFPI3, DGAT2L6, ALOX5, BACH2, CYP4F11, CYP4A22, B3GALT2, or any combination thereof.

In some embodiments, the method comprises reducing or decreasing the expression level of B3GALT1.

In some embodiments, the method comprises increasing the expression level of: IL6, CCL2, CXCL1, TNFPI3, DGAT2L6, ALOX5, BACH2, CYP4F11, CYP4A22, B3GALT2, or any combination thereof, and reducing or decreasing the expression level of B3GALT1.

In some embodiments, the method comprises increasing the expression level of: OR52N, OR5D15P, OR52B6, OR1F1, OR2L3, OR2V2, OR51AB1P, OR1B1, OR4C12, OR6C76, OR2AH1P, OR7A18P, OR1N2, OR10B1P, OR11L1, or any combination thereof.

In some embodiments, the method comprises reducing or decreasing the expression level of: OR5BA1P, OR5M11, OR9A4, OR4U1P, or any combination thereof.

In some embodiments, the method comprises increasing the expression level of: OR52N, OR5D15P, OR52B6, OR1F1, OR2L3, OR2V2, OR51AB1P, OR1B1, OR4C12, OR6C76, OR2AH1P, OR7A18P, OR1N2, OR10B1P, OR11L1, or any combination thereof, and reducing or decreasing the expression level of: OR5BA1P, OR5M11, OR9A4, OR4U1P, or any combination thereof.

In some embodiments, reducing or decreasing comprises at least 5%, at least 15%, at least 25%, at least 35%, at least 50%, at least 75%, at least 85%, at least 95%, at least 99%, or 100% decrease or reduction, or any value and range therebetween. Each possibility represents a separate embodiment of the invention.

In some embodiments, enhancing or increasing comprises at least 5%, at least 25%, at least 50%, at least 75%, at least 100%, at least 150%, at least 250%, at least 500%, at least 750%, or at least 1,000% increase or enhancement, or any value and range therebetween. Each possibility represents a separate embodiment of the invention.

In some embodiments, the cell is a fibroblast. In some embodiments, the cell is a keratinocyte. In some embodiments, the cell is an epithelial cell. In some embodiments, the cell comprises a plurality of cells. In some embodiments, the plurality of cells comprises any combination of: a fibroblast, a keratinocyte, and an epithelial cell. In some embodiments, the cell is a cell of a subject, as described herein.

### Composition

According to some embodiments, there is provided a composition comprising: phytoene in the amount of 55-65% (w/w) of the total carotenoids in the composition, phytofluene in the amount of 10-20% (w/w) of the total carotenoids in the composition, zeta carotene in the amount of 15-25% (w/w) of the total carotenoids in the composition, for use in the treatment of a subject afflicted with a skin barrier related disease.

In some embodiments, the composition comprises phytoene, phytofluene, and zeta carotene. In one embodiment, the composition comprises phytoene, phytofluene, zeta carotene, and an additional carotenoid. In one embodiment, the composition comprises phytoene, phytofluene, zeta carotene, lycopene, beta carotene, gamma carotene, tocopherol, and phytosterol.

In some embodiments, a carotenoid is a natural carotenoid extracted, isolated or purified from a fruit, a vegetable, or a plant (including a plant part). In another embodiment, a carotenoid is carotenoid extracted from a tomato plant. In another embodiment, a carotenoid is a carotenoid extracted from a tomato fruit. In another embodiment, a tomato carotenoid is a tomato extract enriched for a carotenoid. In another embodiment, tomato carotenoid is a carotenoid-rich tomato extract which is all-natural. In another embodiment, tomato carotenoid is a tomato carotenoid complex. In another embodiment, tomato carotenoid complex comprises a complex of phytonutrients including a plurality of carotenoids (such as phytoene, phytofluene, zeta carotene, beta-carotene, etc.), tocopherols and phytosterols. In some embodiments, a carotenoid is a synthetic carotenoid.

In some embodiments, the present invention provides a tomato extract obtained by an innovative extraction protocol. This particular extract which comprises phytoene, phytofluene, and zeta carotene (in amounts as specified hereinbelow), has reduced cellular toxicity. In some embodiments, reduced cellular toxicity is compared to other tomato extracts. In some embodiments, reduced toxicity enables to provide the composition of the invention to a subject in need, at a higher dose without reducing the survival, wellbeing, or both, of the subject. In some embodiments, administering the composition of the invention to a subject in need enables to increase the efficacy of the treatment by providing the active ingredients, such as phytoene, phytofluene, and zeta carotene, at higher amounts which increase the therapeutic effect, but without reducing the survival, wellbeing, or both, of the subject due to high cellular toxicity.

In some embodiments, the composition of the invention provides greater amounts of carotenoids with reduced toxicity compared to other plant-, fruit-, or vegetable-derived extracts, such as a tomato. In some embodiments, the composition of the invention provides increased therapeutic efficacy with reduced toxicity compared to other plant-, fruit-, or vegetable-derived extracts, such as a tomato.

In some embodiments, the composition of the invention comprises natural carotenoids, synthetic carotenoids, or any combination thereof.

In some embodiments, the composition comprises phytoene in the amount of 10-40% (w/w), 15-35% (w/w), 20-45% (w/w), 25-35% (w/w), 20-30% (w/w), or 30-50 (w/w) of the total carotenoids of the composition. Each possibility represents a separate embodiment of the invention.

In some embodiments, the composition comprises phytofluene in the amount of 1-10% (w/w), 3-12% (w/w), 4-14% (w/w), 5-10% (w/w), 8-15% (w/w), or 2-9% (w/w) of the total carotenoids of the composition. Each possibility represents a separate embodiment of the invention.

In some embodiments, the composition comprises zeta carotene in the amount of 4-20% (w/w), 6-18% (w/w), 5-15% (w/w), 6-12% (w/w), 9-17% (w/w), or 10-17% (w/w) of the total carotenoids of the composition. Each possibility represents a separate embodiment of the invention.

In some embodiments, the weight ratio of phytoene and phytofluene combined to zeta carotene ranges from 20:1 (w/w) to 3:1 (w/w), 15:1 (w/w) to 3:1 (w/w), 20:1 (w/w) to 6:1 (w/w), 15:1 (w/w) to 2:1 (w/w), 17:1 (w/w) to 4:1 (w/w), 16:1 (w/w) to 7:1 (w/w), 13:1 (w/w) to 8:1 (w/w), 10:1 (w/w) to 3:1 (w/w), or 15:1 (w/w) to 10:1 (w/w). Each possibility represents a separate embodiment of the invention.

In some embodiments, the composition further comprises an additional carotenoid. As used herein, “an additional carotenoid” refers to any carotenoid or a metabolite thereof, other than or being different from phytoene, phytofluene, and zeta carotene.

In some embodiments, the additional carotenoid is selected from lycopene, beta carotene, gamma carotene, and any combination thereof.

In some embodiments, the composition comprises lycopene in the amount of less than 10% (w/w), less than 7% (w/w), less than 5% (w/w), less than 3% (w/w), less than 2% (w/w), or less than 1% (w/w), of the total carotenoids in the composition, or any value and range therebetween. Each possibility represents a separate embodiment of the invention. In some embodiments, the composition comprises lycopene in the amount of 1-3% (w/w), 1-5% (w/w), 2-6% (w/w), 0.5-4.5% (w/w), 0.1-3% (w/w), 0.6-4.8% (w/w), or 2.5-4% (w/w) of the total carotenoids in the composition. Each possibility represents a separate embodiment of the invention.

In some embodiments, the composition comprises beta carotene in the amount of less than 10% (w/w), less than 7% (w/w), less than 5% (w/w), less than 3% (w/w), less than 2% (w/w), or less than 1% (w/w), of the total carotenoids in the composition, or any value and range therebetween. Each possibility represents a separate embodiment of the invention. In some embodiments, the composition comprises beta carotene in the amount of 1-3% (w/w), 1-5% (w/w), 2-6% (w/w), 0.5-4.5% (w/w), 0.1-3% (w/w), 0.6-4.8% (w/w), or 2.5-4% (w/w) of the total carotenoids in the composition. Each possibility represents a separate embodiment of the invention.

In some embodiments, the composition comprises gamma carotene in the amount of at least 0.15% (w/w), at least 0.18% (w/w), at least 0.2% (w/w), at least 0.25% (w/w), at least 0.35% (w/w), at least 0.5% (w/w), at least 0.75% (w/w), at least 0.9% (w/w), at least 1% (w/w), at least 1.2% (w/w), at least 1.35% (w/w), or at least 1.7% (w/w) of the total carotenoids in the composition, or any value and range therebetween. Each possibility represents a separate embodiment of the invention. In some embodiments, the composition comprises gamma carotene in the amount of 0.15-3% (w/w), 0.2-2% (w/w), 0.2-1.5% (w/w), 0.5-3% (w/w), 0.7-1.6% (w/w), 0.4-2.8% (w/w), or 1.2-3.2% (w/w) of the total carotenoids in the composition. Each possibility represents a separate embodiment of the invention.

In some embodiments, the composition comprises lycopene in the amount of less than 5% (w/w) of the total carotenoids in the composition, beta carotene in the amount of less than 5% (w/w) of the total carotenoids in the composition, gamma carotene in the amount of 0.2-1.5% (w/w) of the total carotenoids in the composition, or any combination thereof.

In some embodiments, the composition comprises an additional carotenoid in the amount of 0.1-3% (w/w), 0.2-3.5% (w/w), 0.5-2.5% (w/w), 0.15-1.75% (w/w), 0.35-2.75% (w/w), 0.8-4% (w/w), 1-5% (w/w), or 1.5-4.75% (w/w). Each possibility represents a separate embodiment of the invention.

In some embodiments, lycopene in the amount of less than 5% (w/w) of total carotenoids in the composition.

In some embodiments, the composition comprises a total carotenoids amount of 5-25% (w/w), 10-15% (w/w), 12-35% (w/w), 3-17% (w/w), 2-20% (w/w), or 1-30% (w/w) of the composition. Each possibility represents a separate embodiment of the invention.

In some embodiments, the composition further comprises a tocopherol (e.g., vitamin E). In some embodiments, the composition comprises a tocopherol in the amount of 1-30% (w/w), 3-35% (w/w), 5-25% (w/w), 2-20% (w/w), 4-41% (w/w), 8-32% (w/w), or 13-39% (w/w) of the composition. Each possibility represents a separate embodiment of the invention.

In some embodiments, the weight ratio of phytoene and phytofluene combined to a tocopherol ranges from 20:1 (w/w) to 3:1 (w/w), 15:1 (w/w) to 3:1 (w/w), 20:1 (w/w) to 6:1 (w/w), 17:1 (w/w) to 4:1 (w/w), 16:1 (w/w) to 7:1 (w/w), 13:1 (w/w) to 8:1 (w/w), 10:1 (w/w) to 3:1 (w/w), or 15:1 (w/w) to 10:1 (w/w). Each possibility represents a separate embodiment of the invention.

In some embodiments, the weight ratio of zeta carotene to a tocopherol ranges from 3:1 (w/w) to 1:3 (w/w), 3:1 (w/w) to 1:2 (w/w), 3:1 (w/w) to 1:1 (w/w), 2:1 (w/w) to 1:1 (w/w), 2:1 (w/w) to 1:2 (w/w), 2:1 (w/w) to 1:3 (w/w), 1:1 (w/w) to 1:2 (w/w), or 1:1 (w/w) to 1:3 (w/w). Each possibility represents a separate embodiment of the invention.

In some embodiments, the composition further comprises a phytosterol.

In some embodiments, the phytosterol is selected from: Cholesterol Brassicasterol, Campesterol, Stigmasterol, β-Sitosterol, Δ5-Avenasterol, Δ7-Avenasterol, Δ7-Stigmasterol, and any combination thereof.

In some embodiments, the composition comprises a phytosterol in the amount of 1-20% (w/w), 2-19% (w/w), 10-25% (w/w), 5-25% (w/w), 8-16% (w/w), 6-18% (w/w), 3-20% (w/w), 4-17% (w/w), or 5-15% (w/w), of the composition. Each possibility represents a separate embodiment of the invention. Each possibility represents a separate embodiment of the invention.

In some embodiments, the weight ratio of phytoene and phytofluene combined to a phytosterol ranges from 20:1 (w/w) to 3:1 (w/w), 15:1 (w/w) to 3:1 (w/w), 20:1 (w/w) to 6:1 (w/w), 17:1 (w/w) to 4:1 (w/w), 16:1 (w/w) to 7:1 (w/w), 13:1 (w/w) to 8:1 (w/w), 10:1 (w/w) to 3:1 (w/w), or 15:1 (w/w) to 10:1 (w/w). Each possibility represents a separate embodiment of the invention.

In some embodiments, the weight ratio of zeta carotene to a phytosterol ranges from 6:1 (w/w) to 2:1 (w/w), 5:1 (w/w) to 2:1 (w/w), 4:1 (w/w) to 2:1 (w/w), 3:1 (w/w) to 2:1 (w/w), 6:1 (w/w) to 3:1 (w/w), 5:1 (w/w) to 3:1 (w/w), 4:1 (w/w) to 3:2 (w/w), or 6:1 (w/w) to 3:1 (w/w). Each possibility represents a separate embodiment of the invention.

Methods for determining the amounts of phytonutrients, such as carotenoids, are common and would be apparent to one of ordinary skill in the art. Non-limiting examples for such methods include, but are not limited to, gas chromatography, liquid chromatography, and mass spectrometry.

In some embodiments, the composition is an oral composition or a topical composition. In some embodiments, the composition is a pharmaceutical or a nutraceutical composition. In some embodiments, the composition comprises a pharmaceutical or a nutraceutical acceptable carrier or excipient. In some embodiments, the composition is a cosmeceutical composition. In some embodiments, the composition comprises a cosmeceutical acceptable excipient. In some embodiments, a composition as described herein in an ingestible skin composition.

In some embodiments, an oral composition is in the form of a soft gel capsule. In some embodiments, an oral composition is in the form of a beverage, a shot, a gummy, or a powder. In some embodiments, an oral composition is mixed or assimilated into a food stuff, such as chocolate, ice cream, or others.

In some embodiments, the composition of the invention is for use in the improvement of skin barrier in a subject in need thereof. In some embodiments, the composition of the invention is for use in the treatment or prevention or a skin barrier related disease in a subject in need thereof. In some embodiments, the composition of the invention is for use in increasing the amount, concentration, or both of linoleate-containing acylceramide in stratum corneum, in a subject in need thereof.

In one embodiment, the composition of the invention can be provided to the individual per-se. As used herein, the term “per-se” refers to a case wherein the composition is not further formulated and or admixed with a carrier, a diluent, etc.

In one embodiment, the composition of the present invention can be provided to the individual as part of a pharmaceutical composition or a nutraceutical composition comprising a pharmaceutically acceptable carrier.

In one embodiment, a “pharmaceutical composition”, a “cosmeceutical composition” or a “nutraceutical composition” refers to a preparation of a composition as described herein with other chemical components such as physiologically suitable carriers and excipients. The purpose of a pharmaceutical composition, cosmeceutical composition, or a nutraceutical composition is to facilitate administration of the composition to an organism.

In some embodiments, a process for producing a composition comprising phytoene in the amount of 20-30% (w/w), phytofluene in the amount of 5-10% (w/w) of, zeta carotene in the amount of 5-15% (w/w), and an acceptable carrier, is provided. In some embodiments, the process comprises extracting a golden tomato as disclosed herein. In some embodiments, a composition of the invention comprises a golden tomato extract produced by the herein disclosed process.

In one embodiment, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

In one embodiment, the phrases “physiologically acceptable carrier” and “pharmaceutically acceptable carrier” which be interchangeably used refer to a carrier or a diluent that does not cause significant irritation to a mammal and does not abrogate the biological activity and properties of the administered composition. An adjuvant is included under these phrases.

In one embodiment, “excipient” refers to an inert substance added to a composition to further facilitate administration of an active ingredient. In one embodiment, excipients include calcium carbonate, calcium phosphate, various sugars and types of starch, cellulose derivatives, gelatin, vegetable oils and polyethylene glycols.

Techniques for formulation and administration of drugs are found in “Remington's Pharmaceutical Sciences,” Mack Publishing Co., Easton, PA, latest edition, which is incorporated herein by reference in its entirety.

In one embodiment, suitable routes of administration, for example, include oral, rectal, transmucosal, transnasal, intestinal or parenteral delivery, including intramuscular, subcutaneous and intramedullary injections as well as intrathecal, direct intraventricular, intravenous, intraperitoneal, intranasal, or intraocular injections.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise” or variations such as “comprises” or “comprising,” indicate the inclusion of any recited integer or group of integers but not the exclusion of any other integer or group of integers.

As used herein, the term “consists essentially of”, or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises”, “comprising”, “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6^{th} Edition) by Leroy “Skip” G Wade; Organic Chemistry by T. W. Graham Solomons and, Craig Fryhle.

### Material and Methods

Lumenato™ (see table 1 for compounds list) was provided by Lycored Corp (Branchburg NJ, USA). Tetrahydrofuran (THF) was purchased from Sigma Chemical Company (St. Louis MO, USA). Primary human dermal fibroblasts derived from the foreskin of a single donor, DMEM, fetal bovine serum and the XTT cell viability kit were purchased from Thermo Fisher Scientific (Branchburg, NJ, USA). The Qiagen RNeasy kit for RNA extraction was purchased from Qiagen (Germantown, MD, USA) A 0.1% (w/v) stock solution of dried tomato extract in THF was prepared for use in the gene expression experiment. Lumenato™ (110 mg) in soft gel capsules was used for the clinical study. The control capsules for this study contained paraffin oil instead of tomato extract.

TABLE 1
Compounds of the golden tomato extract (GTE;
also termed herein Lumenato ™)

| Compound | Weight % |
|---|---|
| Lycopene | 0.55% |
| Tocopherols | 11.27% |
| 15-cis-Phytoene | 24.06% |
| Phytofluene | 6.75% |
| beta-carotene | 0.61% |
| zeta-carotene | 7.48% |
| gamma-carotene | 0.16% |
| Phytosterols | 3.15% |
| Total carotenoids | 39.61% |
| Phytoene and Phytofluene (PP) | 30.81% |

### Gene Expression

The effects of Lumenato™ extract at two concentrations: 0.001% and 0.0005% on gene expression in cultured human dermal fibroblasts was assessed using a microarray analysis.

### Cytotoxicity Assay

Cytotoxicity was evaluated using the XTT Cell Viability Assay (Thermo Fisher Scientific) according to manufacturer's instructions. Fibroblasts in DMEM containing 10% fetal bovine serum were seeded in 96-well plate and cultured overnight at 37° C. with 5% CO₂ and 95% relative humidity. The cells were incubated for 24 hours in the presence of THF at 0.001% (v/v) and 0.0005% and THF plus 0.001% (w/v) Lumenato™ and THF plus 0.0005% Lumenato™, and then incubated in the cell culture medium for additional 24 hours. Untreated cells were included as a negative control. All treatments were performed in triplicates. Inhibition of viability by more than 20% of the control values was considered cytotoxic. The test compounds were not toxic.

### Treatments of Human Dermal Fibroblasts

Human dermal fibroblasts (HDF) were cultured in 6-well plates for 24 hours (Thermo Fisher Scientific/Nunc) and then treated with THE or Lumenato™ for 6 hours. After incubation, cells were washed in PBS and lysates were prepared for total RNA extraction using a Qiagen RNeasy kit according to manufacturer's instructions. RNA concentration and purity were determined using a Nanodrop IMPLEN spectrophotometer (Westlake Village CA, USA).

### Quality Control of Total RNA

RNAs isolated from epidermal tissues were shipped to Advanced BioMedical Laboratories (Cinnaminson, NJ, USA) on dry ice for processing. The overall integrity of total RNA samples and RNA quality was confirmed by Advanced BioMedical Laboratories (Cinnaminson, NJ, USA). A proprietary algorithm that takes several QC parameters into account (e.g., ribosomal RNAs, 28S/18S peak area ratios, unexpected peaks in the 5S region, etc.) was used to calculate the RNA Integrity Numbers (RIN). A RIN number of 10 indicates perfect RNA quality; a RIN number of 1 indicates degraded RNA. According to published data and the inventors own experience, RNA with a RIN number >8 is of sufficient quality for gene expression profiling experiments. RIN number for all RNA samples was >8. Microarray Analysis was performed using Affymetrix Human Clariom D array processing.

### Statistical Analysis of Gene Expression Data

The differential gene expression was obtained using a threshold of 0.05 for statistical significance (p-value) and a log fold change of expression with absolute value of at least 0.6. The fold increase or decrease of the ratio gene expression in the treated cells to the control cells is asymmetric around zero. To avoid this problem, the up or down regulation is usually expressed as the base 2 logarithm of the ratio. Therefore, an absolute value of 0.6 represents a 1.5-fold up or down regulation. A value of 1 represents a two-fold up or down regulation.

### Human Subjects

One hundred and fourteen (114) Japanese women aged 35 through 60 years old were screened to find 66 eligible subjects. The eligible subjects were all of Fitzpatrick skin type II or III and Glogau skin classification type II. Other inclusion criteria included ability to understand the study and willingness to comply with avoiding excessive UV exposure and not taking supplements that could interfere with the study. Of the 63 subjects who completed the study, two had violated protocol, and so their data was not analysed. This study was conducted in accord with the Declaration of Helsinki and was approved by Aisei Hospital Ueno Clinical Research Ethics Committee of Japan (IRB #12000071). All subjects gave written informed consent before the start of the study. Subjects were to make clinic visits at baseline, 4 weeks, 8 weeks and 12 weeks for photography, subjective assessments and instrumental measurements. The 12-week clinical visit was cancelled due to the COVID-19 pandemic and was conducted on week 16 instead. Subjects were to take one Lumenato™ capsule per day with food.

### Measurement of TEWL

TEWL was measured under standard conditions at the clinic visits. A VapoMeter (Delfin Instruments, Tokyo, Japan) was used. The measurements were made on the left cheek on an area from the outer corner of the eye and on a plane with the small-pointed eminence of the external ear (tragus). For measurement of TEWL subjects were acclimatized in an environmental room at 21° C. and 50% relative humidity for 20 minutes, and then skin measurements were taken. All skin measurements were taken by the same person. The skin measurement position was measured with a ruler in order to have the same position at each testing point.

### Photography and Photography Analysis

Subjects photographs were obtained with the use of VISIA Evolution photography device. Primary end point: parameters of VISIA were obtained with using all VISIA modalities: subject positioning was critical and had to be repeated at each time point. Items such as stool height on which the subject sits, and careful placement of the subject's chin and forehead into the imaging device were maintained. Also, the subject's hair was off their face, jewelry removed, and a black drape used to standardize clothing. Subjects' front, left, and right views were captured with their eyes gently closed.

The images were further analyzed by Canfield Scientific (Canfield Scientific, New Jersey USA) to extract and report numerical values corresponding to various changes in the images as described below.

### Red Areas and Texture

Red areas and texture were detected with detailed analysis of VISIA photographs. For such, the detection was conducted by comparing the contrast of the area relative to its local zone. The captured image was first normalized with respect to the skin's background intensity distribution. Such a process eliminates lighting variation across the region of interest. A contrast threshold is applied to select candidate regions which are darker than a minimum intensity level difference, relative to their surroundings. Finally, size and shape criteria are applied to filter the candidate regions and select the final spots.

### Pore Size

Pore size was detected with detailed analysis of VISIA photographs. The pores analysis algorithm provides quantitative data and scoring for visible facial pores. Pores are detected using an image analysis algorithm designed to detect circular objects within a specified diameter range, which also meet a minimum circularity threshold, as well as a minimum gradient threshold (comparing the intensity of the pore relative to its immediate surroundings). The analysis is performed on a high-resolution image captured with a Canfield research capture device (Canfield Scientific, Inc., New Jersey USA).

### Example 1

### Gene Expression Analysis

In the present study, 97 genes were found to be significantly upregulated by the treatment of human dermal fibroblasts with 0.001% Lumenato™ for 6 hrs. Of these, for 55 genes, there was no readily available information relating skin. Many of these were pseudogenes, and seven were olfactory receptor genes. Fifty-eight (58) genes were significantly downregulated, and there was no readily available information related to skin for 36 of these. With 0.0005% Lumenato™ 121 genes were upregulated, and 44 genes were downregulated. There was some overlap with the findings with 0.001% Lumenato™, but also some differences. Notably, twelve additional olfactory receptor genes were identified. The main genes to be discussed are listed in Table 2, and the olfactory receptor genes are listed in Table 3.

TABLE 2
Genes of interest

| | up or down | | |
|---|---|---|---|
| Gene | regulation | p Value | Concentration |
| IL6 | 0.86 | 0.002 | a |
| CCL2 | 0.809 | 1.73E−04 | a |
| CXCL1 | 0.682 | 8.31E−04 | a |
| TNFPI3 | 0.689 | 0.002 | a |
| DGAT2L6 | 0.658 | 0.026 | a |
| ALOX5 | 0.788 | 0.014 | a |
| BACH2 | 0.662 | 0.006 | a |
| CYP4F11 | 0.736 | 8.50E−04 | a |
| CYP4A22 | 0.689 | 0.015 | b |
| B3GALT1 | −0.756 | 0.003 | a |
| B3GALT2 | 0.861 | 0.002 | b |

Concentration^{a} = 0.001% Lumenato ™.
Concentration^{b} = 0.0005% Lumenato ™.
IL6 and CCL2 were also significantly upregulated by 0.0005% Lumenato ™.
The genes are listed from top to bottom in the order in which they are discussed herein.

TABLE 3
Olfactory receptor genes up or down regulated by Lumenato ™

| | Level of Up or | |
|---|---|---|
| Gene | Down Regulation | p Value |
| OR52N4 | 0.984 | 0.002 |
| OR5D15P | 0.789 | 0.031 |
| OR52B6 | 0.734 | 0.022 |
| OR1F1 | 0.683 | 0.013 |
| OR2L3 | 0.66 | 0.003 |
| OR2V2 | 0.656 | 0.019 |
| OR51AB11P | 0.65 | 0.03 |
| OR1B1 | 0.757 | 0.006 |
| OR4C12 | 0.725 | 0.001 |
| OR6C76 | 0.692 | 0.012 |
| OR2AH1P | 0.665 | 0.003 |
| OR7A18P | 0.66 | 0.001 |
| OR1N2 | 0.626 | 0.03 |
| OR10B1P | 0.607 | 0.036 |
| OR11L1 | 0.604 | 0.01 |
| OR5BA1P | −0.615 | 0.005 |
| OR5M11 | −0.626 | 0.049 |
| OR9A4 | −0.654 | 0.002 |
| OR4U1P | −0.727 | 0.002 |

### Example 2

### A Clinical Study—Effects of Lumenato™ on TEWL

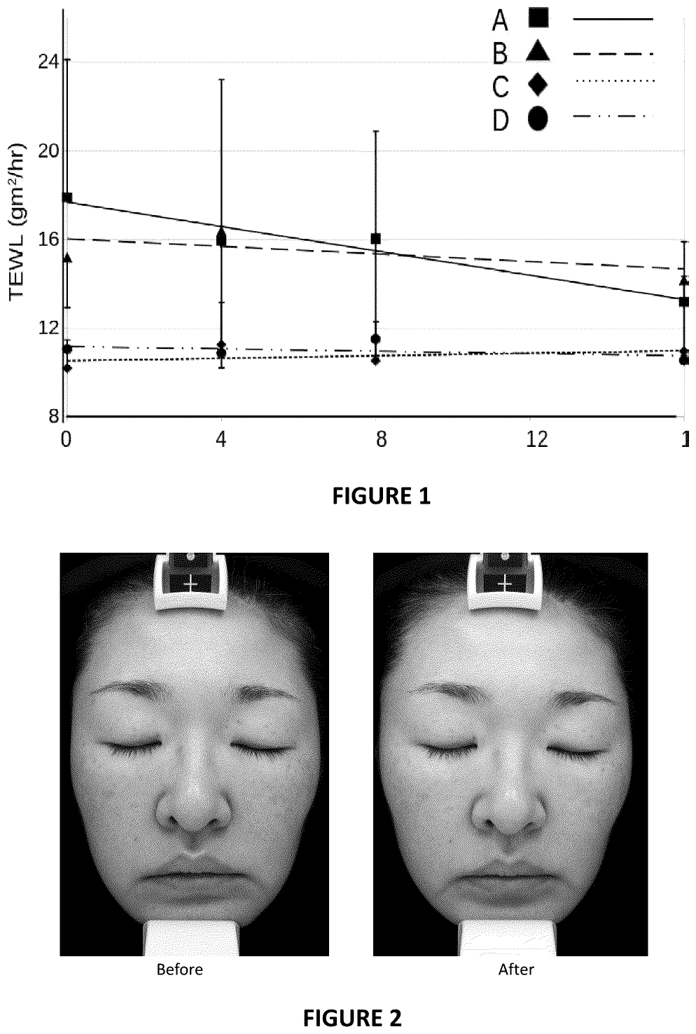

In the clinical study, 32 subjects had relatively high TEWL (value of ≥12.5 g/h/m²) at baseline, while 29 subjects had lower TEWL values. The data from these two groups were analyzed separately. As shown in FIG. 1, Lumenato™ supplementation had no effect on low TEWL. However, in the high TEWL cohort, Lumenato™ supplementation significantly reduced TEWL by 16 weeks.

Calculation of the individual relative change in TEWL for the group that exhibited the significant change ranged between 21 to 53% improvement from baseline. The VISIA photos of these panelists that were further analyzed by Canfield Scientific pointed towards changes in skin appearance: redness, pores and texture. Selected photos are presented herein.

The results represent selected photographs from the supplement sub-group with the TEWL baseline that were analyzed. The analysis reflects fractional areas, meaning, ratio of the total area occupied by the detected feature and the area of interest (area of mask). The detection of features is intensity/contrast-based.

FIG. 2 represents a standard view image of a panelist with 35% improvement in the TEWL value. When comparing the baseline photo to that obtained at week 16 the skin clearly appears smoother and with less apparent pores.

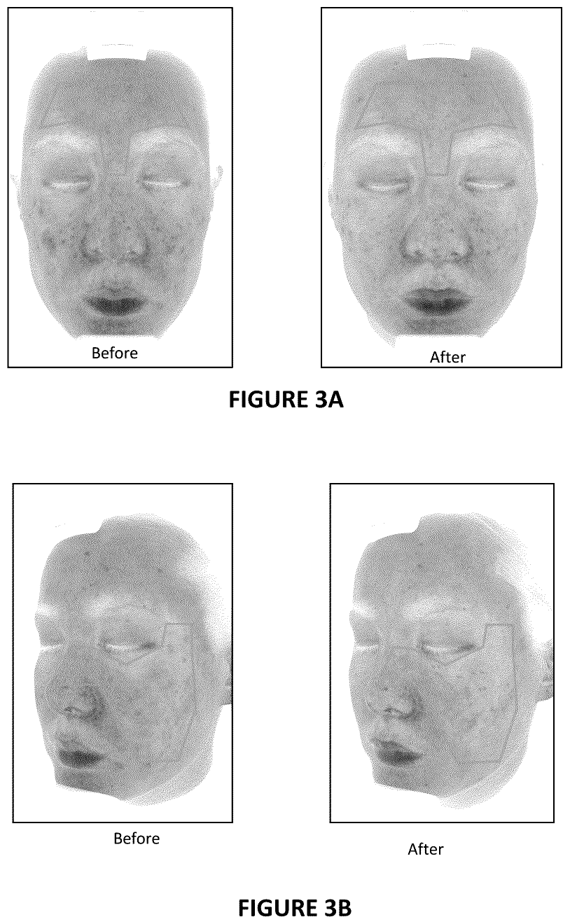

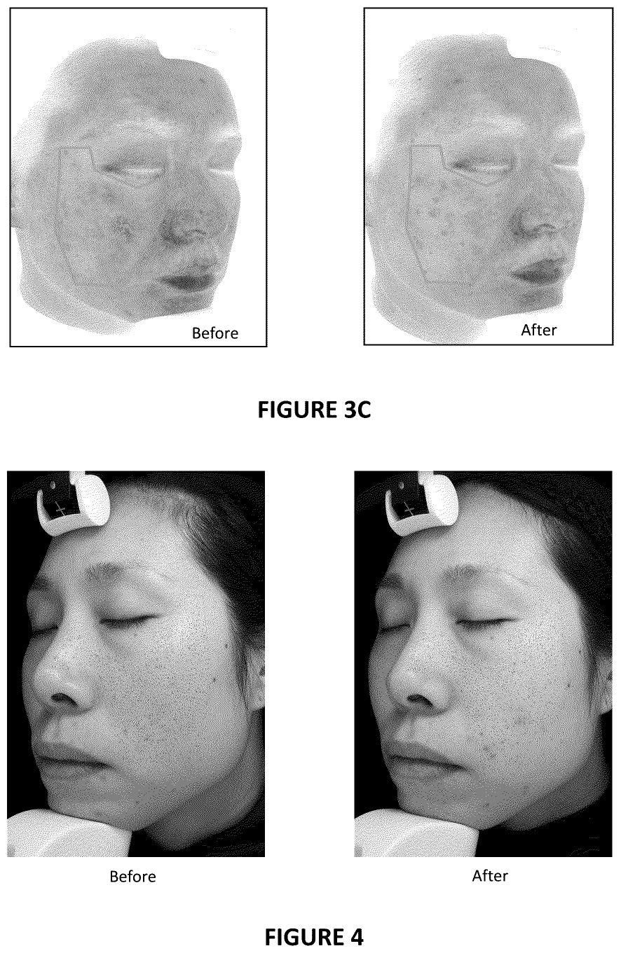

FIGS. 3A-3C represent facial red areas of a panelist with 26% improvement in the TEWL value. When comparing the baseline photo to the one obtained for skin at week 16, a clear reduction in redness is observed.

The red areas represent the presence of blood vessels and hemoglobin in the papillary dermis, a sub-layer of the skin that imparts these structures with the red color, which is detected by RBX Technology (trade name by Canfield scientific) that has been shown to accurately detect and quantify skin erythema. With such, red areas are photographed with cross-polarized lighting and outlined in light blue. Red spots represent erythema and blemishes which are spots while vascular red are linear features. The RBX technology enables devices to observe beneath the skin surface, visualizing specific conditions as related to both vascular disorders and hyperpigmentation. Red areas may be related to a variety of conditions such as inflammation or spider veins. Spider veins or broken capillaries are typically characterized as short, thin lines and can be interconnected in a dense network. Other conditions that result in spots or inflammation vary in size but are generally smaller and round in shape. Large, diffused darker red areas may also indicate an area of concern. The core rationale for this technology lies in the fact that hemoglobin occurs within the vascular structure at the papillary dermis, a sub-layer of skin, in oxygenated and deoxygenated forms and is responsible for red colorations of skin tone. Some skin conditions, such as acne and rosacea, can cause organic changes in the person's vascular structure and elevate the level of hemoglobin present in the dermis. The increased amount of hemoglobin and the formation of new vascular structures will cause a red coloration and will therefore have a negative impact on the evenness of the skin tone. The results presented herein (FIG. 3) present a clear reduction in red area that was numerically calculated at 26% from baseline from the entire face. Interestingly, this panelist also exhibited 26% reduction in TEWL values.

The pores observed in the circular surface openings of sweat gland ducts. Each pore is outlined in dark blue and due to shadowing, enlarged pores appear darker than the surrounding skin tone and are identified by the darker color and circular shape. Outlined areas identify enlarged pores. Previously, the VISIA-CR method for pore assessment was demonstrated to have a high correlation with visual grading and pore size was obtained (r=0.86). In addition, it was demonstrated to be suitable for distinguishing between various age groups in which the shape of the pore and the pore distribution varied.

FIG. 4 presents facial areas of a panelist with 33% improvement in the TEWL value. When comparing the baseline photo to the one obtained at week 16 skin, a clear reduction in the number of pores is observed. This panelist also demonstrated smoother skin.

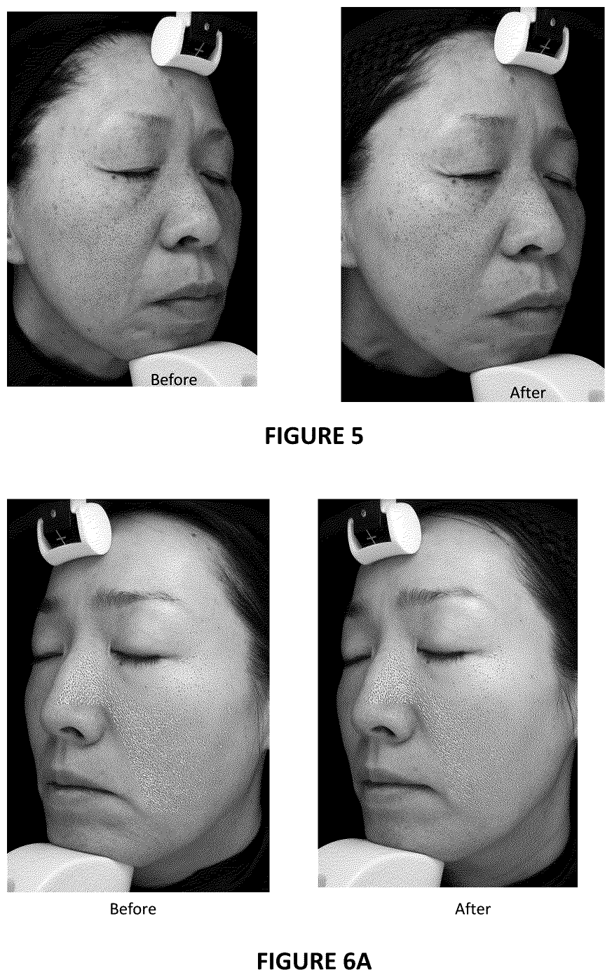

FIG. 5 presents facial red areas of a panelist with 48% improvement in the TEWL value. When comparing the baseline photo to the one obtained at week 16 skin, a clear reduction in number of pores is observed.

In addition to the evaluation of pores, VISIA-CR photography can also allow for the evaluation of changes in skin texture being rougher or smoother. For example, in a study conducted to enhance skin penetration of Rhodobacter sphaeroides extract vis sonophoresis an improvement is skin texture among other attributes was obtained.

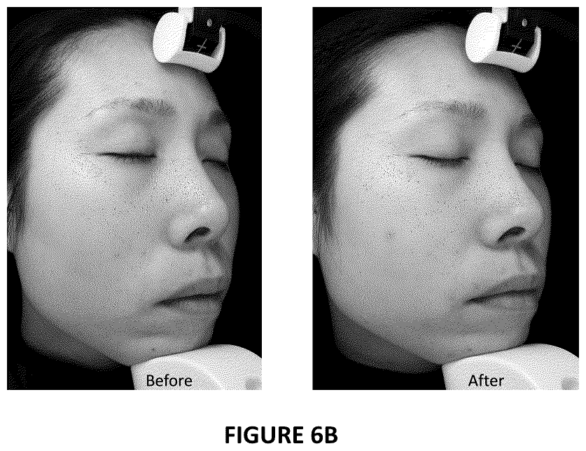

The analysis of texture using VISIA is represented in the 2D photograph by the appearance of raised and depressed areas that indicate variations on the skin surface affecting skin smoothness. As such, raised areas are in yellow and depressed areas are in blue (FIG. 6).

FIGS. 6A-6B present facial red areas of a panelist with 21% improvement in the TEWL value and panelist with 33% improvement in the TEWL value, respectively. When comparing the baseline photos to the ones obtained at week 16 skin, a clear smoothening of the skin texture was observed.

### DISCUSSION

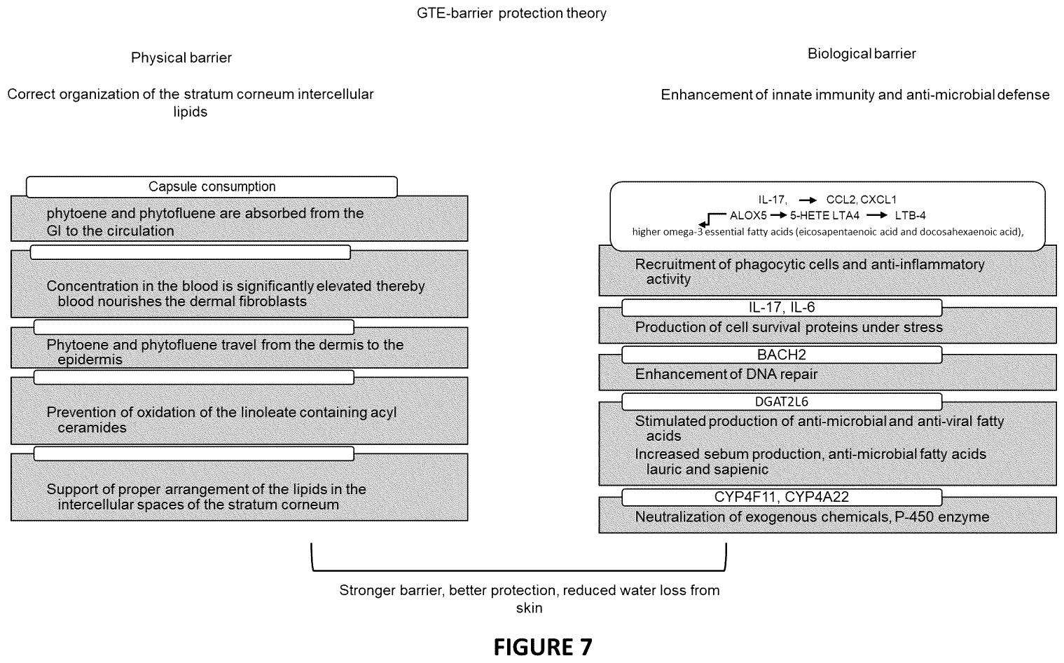

Topical products that address various skin ailments and disorders have been in use since ancient times. In recent decades, with the acknowledgment that the skin is nourished from the blood and with the belief that health equates beauty, the cosmetic and personal care industries are experiencing growth in nutritional supplements that claim improvement in skin appearance of various properties such as wrinkles, age spots and glow. Taken together, the in vitro and in vivo studies for the evaluation of the effect of Lumenato™ on skin indicate that Lumenato™ affects the skin barrier and integrity in two complementary modalities: biological (innate immunity) and physical (the stratum corneum barrier). An illustration describing these two modalities is sketched in FIG. 7.

A number of the upregulated genes appear to enhance dermal defensive mechanisms. These mechanisms include enhancement of innate immunity by recruitment of phagocytic cells and stimulated production of antimicrobial fatty acids, enhanced DNA repair and enhanced detoxification of xenobiotics.

IL-17 expression was not upregulated after 6 hours of Lumenato™ treatment. Presumably it was upregulated at an earlier time, leading to upregulation of some of the genes in the IL-17 pathway at 6 hours. These include IL-6, CCL2, and CXCL1. CCL2 & CXCL1 are chemokine genes which lead to attraction of neutrophiles and monocytes. IL-17 and IL-6 act synergistically to increase production of cell survival molecules under conditions of stress. Likewise, TNF-α is not upregulated at 6 hrs, but TNFIP3 (TNF α-induced protein 3) is upregulated indicating the TNF gene was upregulated earlier. TNF-alpha stimulates collagen synthesis by dermal fibroblasts, which could inhibit formation of or possibly ameliorate wrinkles. TNFIP3 is a deubiquinating enzyme. It removes ubiquitin from cadherin to prevent degradation of cadherin. Collectively, these observations indicate that Lumenato™ consumption activates both the IL-17 pathway and TNF-α pathway in dermal fibroblasts leading to enhanced antimicrobial defense. These two pathways act synergistically in activating antimicrobial defenses.

The gene product of DGAT2L6 increases sebum production. This could make lauric acid and sapienic acid more available at the skin surface, and these are antimicrobials.

Upregulated ALOX 5 protein acts upon arachidonic acid to produce 5-hydroperoxyeicosatetraenoic acid, which can then be converted to 5-hydroxyeicosatetraenoic (5-HETE) acid or to leucotriene A4 (LTA4). LTA4 is rapidly converted to leucotriene B4 (LTB4). 5-Hydroxyeicosatetraenoic acid can be oxidized to 5-oxoeicosatetraenoic acid. LTB4, 5-HETE and 5-oxoeicosatetrenoic acid act as chemotactic factors to attract neutrophils and monocytes, thereby enhancing innate immunity. ALOX5 can also act on the higher omega-3 essential fatty acids (eicosapentaenoic acid and docosahexaenoic acid), and some of these products may be anti-inflammatory.

BACH2 was significantly upregulated. The product of this gene is a marker of aging and DNA damage. It has been implicated in B-cell differentiation and homeostasis of the immune system.

CYP4F11 codes for a P450 enzyme that could be protective against exogenous chemicals. This P450 has been shown to hydroxylate a number of drugs. Interestingly, while expression of CYP4F11 was not significantly affected by treatment with 0.0005% Lumenato™ extract, a different P450 gene, CYP4A22, was upregulated at this lower concentration. This enzyme has been shown to w-hydroxylate lauric and myristic acid and to be capable of hydroxylating a variety of aliphatic and aromatic substrates.

A secondary but extraordinary finding of the present study was the upregulation of a series of genes coding for olfactory receptors. Olfactory receptors are G-protein coupled receptors. They have been found in a range of cell types other than olfactory neurons, including epidermal keratinocytes and melanocytes. In one study, an olfactory receptor (OR2AT4) that responds to a synthetic sandalwood odorant was identified in keratinocytes. The interaction of the odorant with the receptor resulted in calcium uptake. A later study examined the role of this receptor in epithelial cells of the human hair shaft. In this case, activation of the receptor resulted in prolonged hair growth ex vivo. In another study, two other olfactory receptors were identified in keratinocytes. OR2A4/7 responded to cyclohexylsalicylate, and OR51B5 responded to isononyl alcohol. Activation of either of these olfactory receptors resulted in increased intracellular calcium. Olfactory receptors have also been detected in melanocytes. This receptor, OR51E2, responds to β-ionone resulting in increased intracellular calcium and stimulated melanin synthesis. β-ionone is the odorous compound from violets. Although olfactory receptors have been reported in 3T3 cells, an embryonic fibroblast-derived cell line, the present study may be the first to identify numerous olfactory receptor genes in human dermal fibroblasts.

It was also noteworthy that two galactosyltransferase genes were affected by Lumenato™. One of these, B3GALT1, was significantly downregulated, while the other, B3GALT2, was upregulated.

Both β-1,3-galactosyltransferases transfer a galactosyl residue from UDP-galactose to a terminal N-acetylglucosamine of a glycoprotein or ganglioside. The product of B3GALT2 can also transfer galactose to a terminal galactose, but the affinity for this substrate is much lower. These alterations in galactosyl transferase activities will alter the carbohydrate presentation at the cell surface. Consequences of this modification are currently unknown.

As noted previously, the lipids of the stratum corneum consist mainly of ceramides, cholesterol and fatty acids. The fatty acids are mostly 20- through 28-carbons long saturated species. Most of the ceramides also have few readily oxidizable functional groups. These lipids are ideal for resisting oxidative damage. The one exception is the linoleate-containing acylceramide. This lipid is readily oxidized on exposure to air if not protected by an antioxidant. Linoleic acid is essential for the barrier function of the skin. Linoleic acid from the circulation is taken up by the epidermis and is initially incorporated into a small pool of triglycerides. It is rapidly transferred to phosphoglycerides and then to an acylglucosylceramide. This is the glucosylated precursor of the linoleate-containing acylceramide. The glucose is β-glycosidically attached to the primary hydroxyl group of the long-chain base. This linoleate-containing acylglucosylceramide is associated with lamellar granules. In the late stages of the keratinization process, the contents of the lamellar granules are extruded into the intercellular space, and glucosylceramides are deglycosylated to produce ceramides, including the linoleate-containing acylceramide. The linoleate-containing acylceramide is essential for barrier function. Isolated linoleate-containing acylceramide is rapidly oxidized on exposure to air. Under optimal conditions, alpha-tocopherol is delivered to the skin surface via sebaceous secretion, and this protects lipids and proteins from oxidation. Other antioxidants are also delivered in sebum; however, alpha-tocopherol is the major one under most circumstances. Under conditions of oxidative stress, such as UV light exposure, squalene, cholesterol and keratins have been shown to become oxidized, and the oxidized keratins were shown to be in the outer stratum corneum. It seems likely that under such compromised conditions, the linoleate-containing acylceramide would also become oxidized. This would compromise the barrier function of the skin and lead to elevated TEWL. This could account for the subjects with higher TEWL at baseline. Although some carotenoids diffuse from the dermis into the epidermis, a major portion of the carotenoids in the dermis are delivered to the skin surface via sebum secretion and/or sweating. This could provide additional protection to the acylceramide leading to restoration of barrier function with reduced TEWL. The present results from FIG. 1 show that after 16 weeks of Lumenato™ supplementation the TEWL subjects have TEWL values lower than the placebo controls. If the linear trend were to continue, an additional 10 weeks of supplementation would have reduced TEWL to the same range as the low-TEWL group. The error bars represent one-half standard deviation with A and C going up and B and D going down.

The stratum corneum lipids are organized into multilamellar structures in the intercellular spaces of the stratum corneum with a major periodicity of 13 nm that is evident in transmission electron micrographs and from X-ray diffraction data. This 13 nm periodicity is observed with reconstituted stratum corneum lipids; however, if the acylceramide is omitted this periodicity is not seen by either electron microscopy or X-ray scattering. Mutations of enzymes that are uniquely involved in acylceramide synthesis result in various autosomal recessive congenital ichthyoses with impaired barrier function. These genes include ELOV4, CYP4F22, PNPLA1 and ABHD5. The product of ELOV4 is a fatty acid elongase which elongates from C26-CoA to C30-CoA and beyond. CYP4F22 codes for the P450 that ω-hydroxylates the very long fatty acids. The product of PNPLA1 transfers linoleate to the ω-hydroxyl group, and the product of ABHD5 is a coactivator of PNPLA1. In addition, in essential fatty acid deficiency, oleate replaces linoleate in the acylceramide and TEWL increases to at least five-fold. In Gaucher disease the β-gluco cerebrosidase that deglycosylates acylglucosylceramide to produce acylceramide is defective. In this situation, the ultrastructure of the intercellular spaces of the stratum is altered, and the TEWL is markedly elevated. These observations reinforce the concept that the linoleate-containing acylceramide is essential for normal barrier function.

Astaxanthin is a carotenoid found in salmon, salmon roe and some shrimp, and it has been reported to have benefits for skin health and homeostasis. In one randomized placebo-controlled study involving 36 male subjects, after 6 weeks of consuming 6 mg of astaxanthin per day TEWL was significantly reduced in the astaxanthin group compared to controls. Astaxanthin was also reported to lead to improvements in minor wrinkles, age spot size, elasticity, hydration and elasticity. In another study, astaxanthin was shown to increase the minimal erythema dose of UV after 9 weeks of astaxanthin supplementation at 4 mg/day. When TEWL was measured at the site of irradiation it was increased at both the control and astaxanthin group, but the increase was significantly less in the astaxanthin group.

A number of studies have shown that when TEWL is reduced by various interventions, hydration and skin surface smoothness increased, and scaliness and pore area decreased. Likewise, in studies in which the skin was irritated by topical application of chemicals or by using harsh soap TEWL and erythema both increased. Thus, TEWL correlates with a number of skin appearance parameters. In the present study, correlation was found between reduction of TEWL and various attributes in skin's appearance that were captured by VISIA photography and qualitatively and quantitively analyzed. Specifically, reduction in skin redness and skin pores and overall improvement in skin texture (reduction in roughness) and appearance has been demonstrated to accompany a reduction in TEWL. Skin redness is a sign of inflammation that may be sub-clinical levels of an underlying disease, a manifestation of primary irritation or an allergic reaction. Since carotenoids are known anti-inflammatory and anti-oxidant agents the attenuation of skin redness may be explained by such activity, but the fact that the reduction in skin redness was strongly associated with reduction in TEWL points towards a potentially more complex multi-functional path. Moreover, the skin barrier is known to maintain a relatively stubborn homeostasis, even if such is abnormal. This means that when challenged by a detergent, abnormal pH or other irritating conditions, in most cases it will bounce back to its baseline within hours to days.

The VISIA photos demonstrate a reduction in concentrated darker red areas. These represent blood vessels and hemoglobin in the papillary dermis, a sub-layer of the skin, give these structures the red color, which is detected by RBX Technology. The red areas may be related to a variety of conditions such as inflammation or spider veins. Spider veins or broken capillaries are typically characterized as short, thin lines and can be interconnected in a dense network.

A correlation between reduction in TEWL and improvement in skin texture was observed with the application of topical retinoids to skin. The similarity in effect may not be surprising since carotenoids are the oxidative precursors for retinoids.

In this study the efficacy and safety of new anti-aging creams containing retinaldehyde were evaluated as treatment for photoaged skin. This study was conducted on 40 female Korean volunteers who applied retinaldehyde cream twice daily for 3 months. Crow's feet wrinkles and skin texture were quantitatively assessed using the Antera 3D® system. TEWL, skin hydration, the melanin index, and skin brightness were also evaluated. The results of this study demonstrated that the treatment improved the condition of photoaged skin.

In summary, the studies presented herein, reveal a unique effect of Lumenato™ on skin at the dermal and epidermal levels. These carotenoids and tocopherols strengthen the skin barrier and equip it with tools to cope with environmental insult. Moreover, the inventor demonstrated a correlation between barrier strength to specific skin appearance related benefits such as reduction in redness and in pores and skin smoothening effect. Further, the inventor demonstrated the existence of olfactory receptors in human dermal fibroblasts and their significant up regulation by Lumenato™.

### Example 3

### Effect of Lumenato Oral Consumption on Improvement of Visual and Experimental Skin Attributes

Healthy women between the ages of 35 and 55 interested in improving health and appearance of normal facial skin were recruited for the study. The subjects downloaded an app (ClaimIt, Obvio Health USA Inc.) which is an online and mobile app interface to execute clinical trials. This setup eliminates physical site visits and brings the trial directly to the mobile device of each subject. The participants signed an electronic informed consent (eIC) before beginning the screening process after which they completed a study-specific screening questionnaire with the inclusion/exclusion criteria, including self-reported ethnicity and skin type. On qualification, they completed medical history and concomitant medication forms and enrolled in the study. The qualified participants were shipped the study product. The participants took 1 softgel each day for 12 weeks around the time of a primary meal (breakfast, lunch, or dinner). They were required to complete questionnaires within the app as they are rolled out at predefined time points throughout the study. The weekly e-diary was used to measure compliance with consuming the study product as well as any reported changes in health or medication to determine adverse event if any. At the end of the study (12 weeks of use), the participants were instructed to safely discard any remaining softgels containing the study product and to complete a series of questionnaires relating to their experience with the study product. Two weeks after completing the treatment period, the participants completed the FACE-QTM Satisfaction with Skin, End of Study Skin Questionnaire, and ClaimIt Experience Survey.

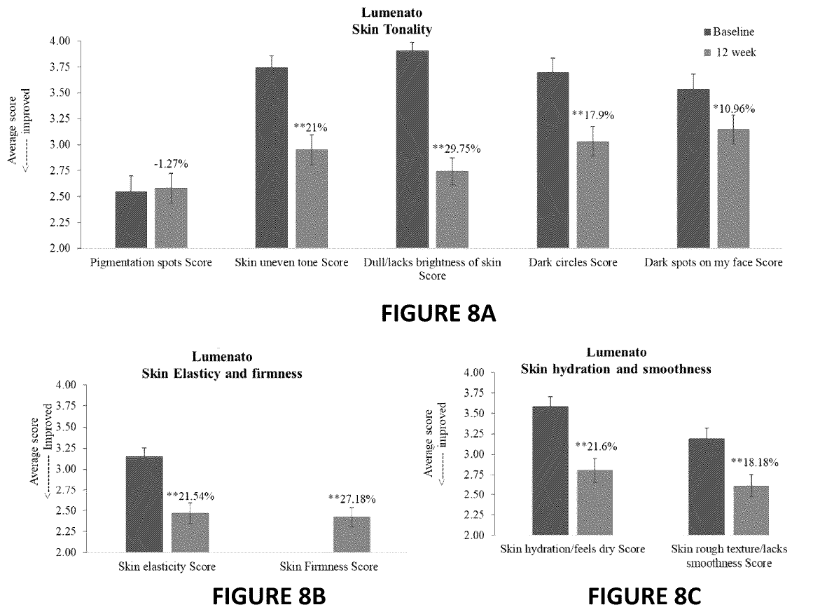

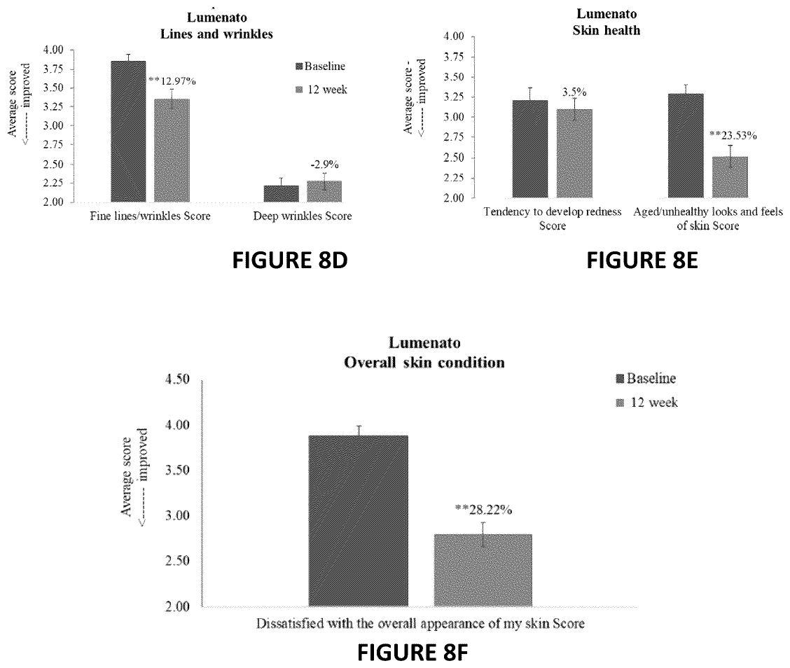

FIG. 8 exhibits the average scores of different parameters before and after 12-week use of the supplement. FIG. 8A shows various measurements of skin tonality. As observed in the graph, although there was no improvement in pigmented spots, there was still a 21% improvement in evenness of skin tone (p<0.001). The skin appeared 29.75% brighter (p<0.001) and dark spots of the face reduced by 10.96% (p<0.001). Periorbital dark circles also appeared to diminish by 17.9% (p<0.001). FIG. 8 exhibits the average scores of different parameters before and after 12-week use of the supplement. As observed in FIG. 8B, there was a highly significant (p<0.001) improvement in skin elasticity (21.54%) and firmness (27.18%) after using the supplement for 12 weeks. FIG. 8A shows various measurements of skin tonality. As observed in the graph, although there was no improvement in pigmented spots, there was still a 21% improvement in evenness of skin (p<0.001). The skin appeared to be 29.75% brighter (p<0.001) and dark spots of the face reduced by 10.96% (p<0.001). Periorbital dark circles also appeared to diminish by 17.9% (p<0.001). FIG. 8C displays that there was 21.6% (p<0.001) perceived improvement in skin hydration and 18.18% improvement in skin texture (p<0.001) after 12 weeks of using the supplement. FIG. 4D shows that although there was no improvement in deep wrinkles, there was a significant improvement of 12.97% (p<0.001) in fine lines and wrinkles of the face after 12 weeks of supplement use. As observed in FIG. 8E, there was a slight improvement in the tendency of the skin to develop redness. The general look of aged/unhealthy skin reduced significantly (p<0.001) after using the supplement with a 23.53% reduction. FIG. 8F shows the average score of overall skin condition before and after 12 weeks of treatment of the supplement. As observed in the graph, there was a highly significant (p<0.001) improvement of 28.22% in the overall skin condition after using the supplement.

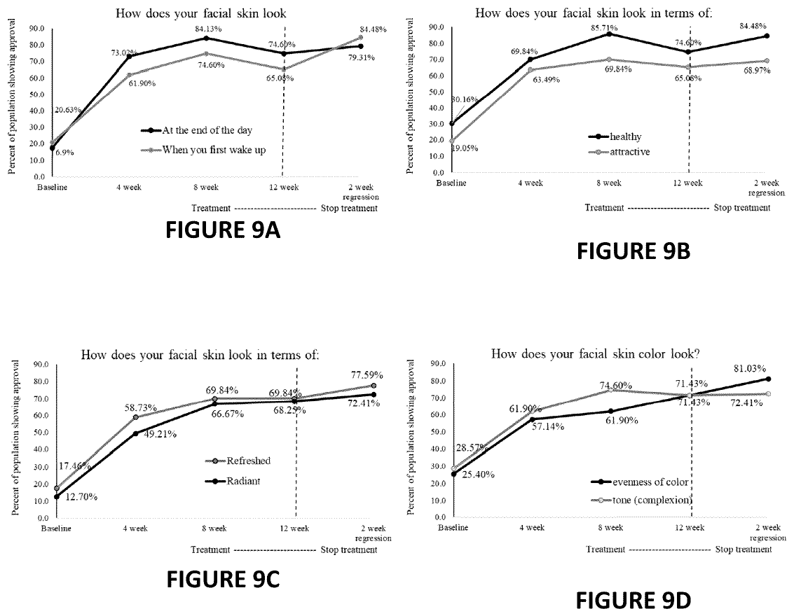

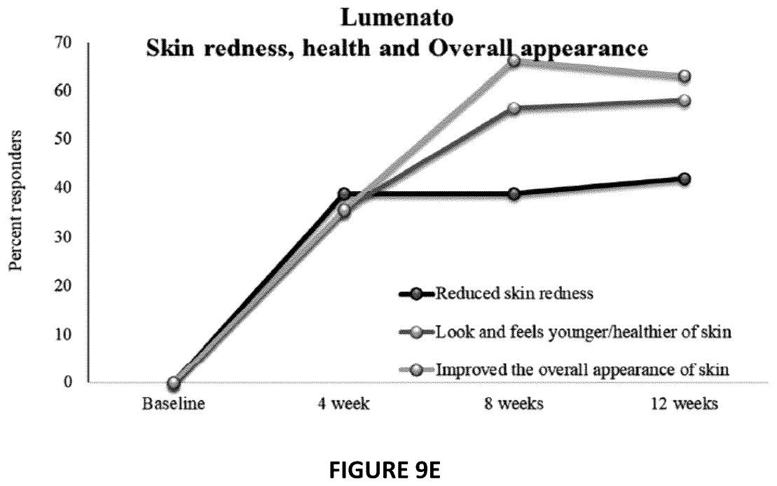

FIG. 5 exhibits results from FACE-QTM Satisfaction with Skin Scale before treatment and after 4, 8, and 12 weeks of treatment and again after treatment was stopped for 2 weeks. The data presented are from satisfaction score where score 1 was very dissatisfied, 2 was somewhat dissatisfied, 3 was somewhat satisfied, and 4 was very satisfied. The number of subjects reporting a score of 3 and 4 was counted and expressed in the graph as percent of population showing approval. FIG. 9A shows “how does your facial skin look” first thing in the morning and at the end of the day. Initially, the score was at the edge of satisfied and dissatisfied; however, after treatment, there was a steady improvement up to eight weeks of 84%-75% (p<0.001), after which there was a levelling off. The effect retained after the treatment was discontinued for two weeks. FIG. 9B shows a similar trend for perception of healthy and attractive look of the face. For these both parameters, there was steady improvement over the course of 12-week treatment at which point there was a statistically significant (p<0.001) improvement of 74.6% and 65.08% (p<0.001) for healthy and attractive skin, respectively. These perceptions continued to improve despite discontinued treatment for two weeks.

FIG. 9C: Perception of refreshed and radiant face. For these two parameters, there was steady improvement over the course of 12 weeks of treatment at which point there was a statistically significant (p<0.001) change as compared to baseline. These perceptions continued to improve despite discontinued treatment for two weeks.

FIG. 9D: facial skin tonality. After treatment, there was a steady improvement up to eight weeks of 61.9%-74.6% (p<0.001) in evenness of color and tonality. By the 12^{th} week of treatment, over 71% (p<0.001) of the population professed that the treatment was effective for skin tonality and evenness of tone. After treatment was discontinued for two weeks, there was a further improvement of 81% and 72.41% in evenness of skin tone and tonality, respectively.

FIG. 9E shows that 38.71% of the population noticed a reduction in skin redness after using the supplement for 4 and 8 weeks, increasing to 41.94% after 12 weeks of supplement use. Improvement in the look and feel of healthy skin was observed by 35% of the population after 4 weeks of use increasing to 56.45% and 58.06% after 8 and 12-week use, respectively. The overall appearance of skin improved in 35.48% of the subjects after 4 weeks of use increasing to 66.13% and 62.9% after 8 and 12 weeks, respectively.

Facial lines and wrinkles are the most scrutinized manifestation of aging. It has been reported that the furrows and wrinkles are deeper and denser in the skin of individuals with a low antioxidant level. In the current study, there was a significant reduction in the fine lines and wrinkles after 12 weeks of product use. Most subjects in this study exhibited a very low score for deep wrinkles; nevertheless, 61% of the subject population noticed an improvement in wrinkles after treatment with the supplement.

### Example 4

### A Clinical Study to Evaluate the Efficacy and Consumer Perception of an Oral Supplement

The first objective of this clinical study is to assess the efficacy of an oral supplement to change the characteristics of facial skin after four, 8 and 12 weeks of use. The second objective is to obtain consumer perception of the test product through the use of questionnaires.

Approximately 66 subjects are enrolled in this clinical study to assess the efficacy of an oral supplement to change the characteristics of facial skin after four and 12 weeks of use and obtain consumer perception of the test product through the use of questionnaires. Study evaluations include a comprehensive metabolic panel, Cutometer measurements, Canfield's VISIA-CR images, VapoMeter Measurements, and Consumer perception questionnaires.

### Vapometer

The VapoMeter is the only fully portable instrument available for the measurement of TEWL (transepidermal water loss) values and evaporation rates. TEWL is a well-known indicator of the skin's barrier function. Two VapoMeter measurement are obtained from the randomized cheek at each designated time point.

### Cutometer

The Cutometer is capable of assessing the mechanical properties of the skin in a non-invasive manner. The instrument measures the vertical deformation of the surface of the skin as it is pulled by a vacuum suction (500 mm Hg) through a small probe aperture.

The following parameters can be measured: The final distention, Uf, (R0) measured at 10 seconds; The immediate distention, Ue, measured at 0.1 seconds; The delayed distention, Uv; Immediate retraction, Ur; The deformation parameters are extrinsic parameters dependent on skin thickness. In order to circumvent the measurement of skin thickness, the following ratios are used to evaluate the elastic nature of the skin:
R0: This parameter represents the passive behavior of the skin to force. Decrease in this parameter indicates improvement in firmness.
R1: The ability of the skin to return to its original state. Decrease in this parameter indicates improvement.
R2: Gross elasticity, the closer the value is to 1 (100%) the more elastic the curve, very important parameter. R2 represents the elasticity of the skin and its ability to recover its original position. Increase in this parameter indicate improvement in elasticity.
R3: Last curve, compared to the maximum amplitude of the first curve. Tiring effects of the skin are visible, as the amplitude increases with each new suction. R3 is correlated with skin fatigue. Repeated suction and release with the Cutometer provoked skin fatigue, resulting in decreased elasticity and increased maintenance of the deformed position. Decrease in this parameter indicates improvement.
R4: Last minimum amplitude compared to the first curve, tiring effects of the skin are visible, as the ability of redeformation decreases with each new suction. R4 is correlated with skin fatigue. Repeated suction and release with the Cutometer provoked skin fatigue, resulting in decreased elasticity and increased maintenance of the deformed position. Decrease in this parameter indicates improvement.
Ur/Ue (R5) is the biological elasticity of the skin. It measures the ability of the skin to regain its initial configuration after deformation. A value of 1 would indicate 100% elasticity. Increase in this ratio indicate improvement in elasticity.

Uv/Ue (R6) is the ratio between delayed and immediate deformation, i.e. it is the viscoelastic to elastic ratio. An increase in the value of this ratio indicates that there has been an increase in the viscoelastic portion of the deformation and/or relative decrease of the elastic part. Decrease in these ratio indicate improvement in elasticity.

Ur/Uf (R7) is a measure of the net elasticity of the skin. Increase in this ratio indicate improvement in elasticity.

Cutometer measurements will be obtained for R1, R2, R3, R4, R5, R6, R7, and R0.

Measurements are obtained by holding the probe firmly against the surface of the skin. The vacuum pump suctions the skin into a small opening and the probe measures the height to which the skin is distended.

Measurements are automatically produced in triplicate, by running through three twenty-second cycles. Each cycle is made up of a suction phase, where the skin is sucked into the probe, and a relaxation phase, where the suction stops, and the skin is released. Data is electronically imported into the computer, where ratio values are calculated. Readings are displayed and then manually transcribed onto a score sheet.

One Cutometer measurement will be obtained from the randomized cheek at each designated time point.

### Canfield's VISIA-CR

Canfield's VISIA-CR's sophisticated technique produces high quality, reproducible facial images which can demonstrate treatment efficacy through subjective clinical evaluations of skin features such as wrinkles and fine lines, skin texture, coloration/evenness, photodamage, vascular features and porphyrins (P. acnes).

VISIA-CR imaging is captured with the eyes closed. Subjects will wear a headband to pull back the hair and a cape to cover clothing. Subjects may be asked to remove jewelry at the discretion of the technician.

The following parameters will be captured: Assessment of fine lines and wrinkles (crow's feet, forehead, cheeks), Pores, Dark spots, Pigmentation/Homogeneity, Texture/Roughness, and Porphyrins.

This study is performed on 66 subjects at 3 time points. Analysis is performed on VISIA images for all the subjects.

Specifications of the study are as follows:
66 subjects: 33 in the active group and 33 in the placebo group
2 products (Active vs Placebo)
3 time points: Baseline, Week 4 and Week 12
Left, Face and Right acquisitions
Cross, Parallel, UV and Standard polarizations (All Visia CR modalities)

The measurement and lighting systems must be the same throughout the study. Besides, the subject must be very good repositioned between time points.

### Crow's Feet Analysis

In order to evaluate the product efficacy on the crow's feet, the analysis is performed on Left and Right images.

Definition of a region of interest on each crow's feet area (Left and Right).

Spatial registration of regions between images for each subject in order to follow exactly the same regions over time.

Study and optimization of extraction parameters for crow's feet wrinkles on a few images.

Extraction of crow's feet wrinkles with optimized parameters on all the images.

Extraction of the crow's feet wrinkles parameters: Visible surface; Visible depth; Visible volume; and Visible length.

### Forehead Wrinkles Analysis

In order to evaluate the product efficacy on the forehead wrinkles, the analysis is performed on Face images.

### Definition of a Region of Interest on the Forehead Area

Spatial registration of regions between images for each subject in order to follow exactly the same region over time

Study and optimization of extraction parameters for forehead wrinkles on a few images

Extraction of forehead wrinkles with optimized parameters on all the images

Extraction of the forehead wrinkles parameters: Visible surface; Visible depth; Visible volume; and Visible length.

### Cheek Fine Lines Analysis

In order to evaluate the product efficacy on the cheeks, the analysis is performed on left and right images.

Definition of a region of interest on each cheeks (Left and Right).

Spatial registration of regions between images for each subject in order to follow exactly the same region over time.

Study and optimization of extraction parameters for cheek fine lines on a few images.

Extraction of cheek fine lines with optimized parameters on all images.

Extraction of the cheek fine lines parameters: Visible surface; Visible depth; Visible volume; and Visible length.

### Color and Homogeneity Analysis

In order to evaluate the product efficacy on the skin color and blemish, the analysis is performed on Face, Left and Right images.

Selection of one region on the cheek (right and left profiles) and one region on the forehead (face) on each photo at baseline.

Spatial registration of regions between images for each subject in order to follow exactly the same region over time.

Data extraction of colorimetric parameters: L*, a*, b*, ITA∘ and IWA∘Newtone (whitening index).

### Skin Texture Analysis

In order to evaluate the product efficacy on the skin roughness, the analysis is performed on Face, Left and Right images.

Selection of one region on the cheek (right and left profiles) and one region on the forehead (face) on each photo at baseline.

Spatial registration of regions between images for each subject in order to follow exactly the same region over time.

Haralick extraction parameters: contrast and entropy related to skin smoothness and texture regularity.

Computation of 2D roughness parameters: Spa, Spq and Sdev.

### Pores Analysis

In order to evaluate pores, the analysis is performed on face images.

Definition of a region of interest near the nose on left and right side of the face.

Spatial registration of regions between images for each subject in order to follow exactly the same region over time.

Study and optimization of extraction parameters for pores on a few images.

Extraction of pores with optimized parameters on all images.

Analysis of extracted regions characteristics: Number; Visible surface; Visible depth; and Visible volume.

### Porphyrin Analysis

In order to evaluate porphyrins, the analysis is performed on the upper side of the cheeks (near the nose) on face images.

Definition of one region of interest on the nose and near the nose.

Spatial registration of regions to analyze the same regions over time.

Parameters adjustment for porphyrin detection and segmentation.

Extraction of porphyrin with optimized parameters on all images.

Quality control on all the segmented images.

Analysis of porphyrin: number of elements, quantity and surface of fluorescence.

### Dark Spots Analysis

In order to evaluate the product efficacy on dark spots, the analysis is performed on Left and Right images.

Selection of the overall pigmented spots on each subject on the 2 cheeks at TO.

Detection and automatic segmentation of pigment spots for all the time points.

Extraction of the surface of pigment spots.

Extraction of color (L*, a*, b*, ITA∘, IWANewtone∘) of pigments spots, surrounding skin and contrast between pigment spots and surrounding skin.

### Consumer Perception Questionnaire

An assessment of test material attributes and the effects of a test material can be determined by questioning the treated subject with regard to consumer perception and the efficacy of the test material following use. Questionnaire administration using EvaSys or similar allows for an efficient and accurate method of determining subject response proportions to assess the consensus opinion of a clinical study population.

### Comprehensive Metabolic Panel

Blood is drawn from each subject to be submitted for a comprehensive metabolic panel. The following parameters are included in the comprehensive metabolic panel: glucose, calcium, proteins (albumin and total protein), Electrolytes (sodium, potassium carbon dioxide, and chloride), kidney tests (blood urea nitrogen and creatinine), and liver tests [alkaline phosphatase, alanine amino transferase (also called SGPT), aspartate amino transferase (also called SGOT), and bilirubin].

Phlebotomy procedures are performed in accordance with CRL SOP AC 1.3. The comprehensive metabolic panel provides results on liver and kidney function, to confirm test material safety over the duration of the study. Subjects with values outside of the acceptable range are not enrolled or discontinued.

Blood draws are done by a certified technician at Baseline and Week 4. Samples are sent to a lab for a metabolic panel.

### D-Squame Tape Stripping

The shedding or accumulation of the stratum corneum in flakes is called desquamation. Under normal conditions, the epidermis is completely replaced every 25 to 30 days, depending on the skin site. Corneocytes pile up to the stratum corneum at the end of the process of keratinization. Abnormal shedding produced by underlying conditions like parakeratosis (psoriasis) or by loss of lipids and hydration (xerosis) leads to squamous eruptions. D-Squame is a sampling device, 22 mm diameter, crystal-clear adhesive-coated disc. It has a homogenous layer of a medical-grade adhesive that safely removes superficial corneocytes and provides optimum visibility of adhering skin cells.

### Tape Stripping Method

A series of tape strips are obtained sequentially from the same site to remove several layers of the stratum corneum. Tape-stripping is performed using D-Squame standard sampling discs (circular 22 mm adhesive disc).

Study procedure: Tape stripping is performed on the randomized right or left cheek. A D-Squame standard sampling disc is applied to the randomized cheek then removed 3 consecutive times, using a new piece of tape each time using the method above, TEWL readings are taken following every 3 tape strips. This is repeated until the TEWL reading of 20 g/m²/hr is reached.

### Baseline

Subjects report to the testing facility with clean faces, free from makeup. Photograph Release form is filled out by subjects. Informed consent is obtained. Inclusion and exclusion criteria are verified. Subjects acclimate to ambient laboratory conditions for approximately 15 minutes (±5 minutes).

The following study evaluations are performed: Comprehensive metabolic panel, Cutometer measurements, Canfield's VISIA-CR images, and VapoMeter measurements.

An ECRL technician tape-strips the randomized left or right side of the cheek with D-squame until a TEWL reading of approximately 20 g/m²/hr is reached. Subjects are instructed to return to the laboratory approximately 3 hours post tape-stripping. Subjects are instructed to refrain from washing or applying any products to their face.

### 3 Hours Post-Tape Stripping (±30 Minutes)

Subjects acclimate to ambient laboratory conditions for approximately 15 minutes (±5 minutes). An ECRL technician obtains VapoMeter measurements.

### Week 4

Subjects return to the testing facility with clean faces, free from makeup. Subjects acclimate to ambient laboratory conditions for approximately 15 minutes (±5 minutes). The following study evaluations is performed: Comprehensive metabolic panel, Cutometer measurements, Canfield's VISIA-CR images, VapoMeter measurements, and Consumer perception questionnaire.

An ECRL technician tape-strips the randomized left or right side of the cheek with D-squame until a TEWL reading of approximately 20 g/m²/hr is reached. Subjects are instructed to return to the laboratory approximately 3 hours post tape-stripping. Subjects are instructed to refrain from washing or applying any products to their face.

### 3 Hours Post-Tape Stripping (+30 Minutes)

Subjects acclimate to ambient laboratory conditions for approximately 15 minutes (±5 minutes). An ECRL technician obtains VapoMeter measurements.

### Week 8

Subjects return to the testing facility.

The following study evaluation is performed: Consumer perception questionnaire.

### Week 12 (Final Visit)

Subjects return to the testing facility with clean faces, free from makeup. Subjects acclimate to ambient laboratory conditions for approximately 15 minutes (±5 minutes). Daily diaries are reviewed for study compliance and collected.

The following study evaluations are performed: Cutometer measurements, Canfield's VISIA-CR images, VapoMeter measurements, and Consumer perception questionnaire.

An ECRL technician tape-strips the randomized left or right side of the cheek with D-squames until a TEWL reading of approximately 20 g/m²/hr is reached Subjects are instructed to return to the laboratory approximately 3 hours post tape-stripping. Subjects are instructed to refrain from washing or applying any products to their face.

### 3 Hours Post-Tape Stripping (±30 Minutes)

Subjects acclimate to ambient laboratory conditions for approximately 15 minutes (±5 minutes). An ECRL technician obtains VapoMeter measurements.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method for treating a skin barrier related disease or disorder in a subject in need thereof, comprising the step of administering to said subject a therapeutically effective amount of a composition comprising: phytoene in the amount of 55-65% (w/w) of the total carotenoids in said composition, phytofluene in the amount of 10-20% (w/w) of the total carotenoids in said composition, zeta carotene in the amount of 15-25% (w/w) of the total carotenoids in said composition, thereby treating a skin barrier related disease or disorder in the subject.

2. The method of claim 1, further comprising a selecting step preceding said administering step, comprising determining a transepidermal water loss (TEWL) value of a skin of said subject, wherein a TEWL value of at least 12 (g·h/m²) is indicative of said subject being suitable for said treating.

3. The method of claim 2, wherein said TEWL value is determined for a facial skin, forehead skin, a forearm skin, hand skin, palm skin, leg skin, elbow skin, or any combination thereof, of said subject.

4. The method of claim 1, wherein said skin barrier related disease is an epidermal skin disease or disorder.

5. The method of claim 1, wherein said skin barrier related disease is characterized by alteration of the stratum corneum: lipid content, oxidative state, arrangement, or any combination thereof.

6. The method of claim 1, wherein said skin barrier related disease is characterized by a reduced amount of linoleate-containing acylceramide.

7. The method of claim 1, wherein said skin barrier related disease is selected from the group consisting of: atopic dermatitis, psoriasis, type 2 Gaucher syndrome, Sjogren-Larsson syndrome, lamellar ichthyosis, X-linked ichthyosis, bullous ichthyosiform erythroderma, essential free fatty acid deficiency, acne vulgaris, aged dry skin, hypohidrotic ectodermal dysplasia, and atopic eczema.

8. The method of claim 1, wherein said treating comprises reducing: TEWL, number of pores, area covered by pores, level of redness, area covered by redness, roughness, or any combination thereof, of a skin of said subject.

9. The method of claim 8, wherein said reducing is by at least 5% compared to a control.

10. The method of claim 1, wherein the weight ratio of said phytoene and said phytofluene combined to said zeta carotene ranges from 15:1 (w/w) to 2:1 (w/w).

11. The method of claim 1, wherein said composition further comprises an additional carotenoid selected from the group consisting of: lycopene, beta carotene, gamma carotene, and any combination thereof.

12. The method of claim 11, wherein said composition comprises said lycopene in the amount of less than 5% (w/w) of the total carotenoids in said composition, said beta carotene in the amount of less than 5% (w/w) of the total carotenoids in said composition, said gamma carotene in the amount of 0.2-1.5% (w/w) of the total carotenoids in said composition, or a combination thereof.

13. The method of claim 1, wherein said composition comprises a total carotenoids amount of 10-15% (w/w) of said composition.

14. The method of claim 1, wherein said composition further comprises a tocopherol.

15. The method of claim 14, wherein said composition comprises said tocopherol in the amount of 10-30% (w/w) total carotenoids in of said composition.

16. The method of claim 1, wherein said composition further comprises a phytosterol.

17. The method of claim 16, wherein said composition comprises said phytosterol in the amount of 5-15% (w/w) total carotenoids of said composition.

18. The method of claim 1, wherein said administering comprises orally administering, topically administering, or both.

19. (canceled)
